SEQUENCE LISTING (219 sequences)

SEQ ID NO: 1 - DNA, 54 bp, Artificial Sequence
  Description of Artificial Sequence Integrin
acttacaaaacaaaggaggaaatgatagtagcaacgagtcagaccagtcaatat

SEQ ID NO: 2 - protein, 18 aa, Artificial Sequence
  Description of Artificial Sequence Integrin
TYKTKEEMIVATSQTSQY

SEQ ID NO: 3 - DNA, 15 bp, Artificial Sequence
  Description of Artificial Sequence Integrin
cagaccagtcaatat

SEQ ID NO: 4 - protein, 5 aa, Artificial Sequence
  Description of Artificial Sequence Integrin
QTSQY

SEQ ID NO: 5 - DNA, 15 bp, Artificial Sequence
  Description of Artificial Sequence Integrin
atagcagtaatagga

SEQ ID NO: 6 - protein, 5 aa, Artificial Sequence
  Description of Artificial Sequence Integrin
IAVIG

SEQ ID NO: 7 - DNA, 261 bp, Artificial Sequence
  Description of Artificial Sequence Integrin
aatttcctagagaagtttgttcagggtctcgatatcggccctaccaaaacccaggtcggtctgatacaatatgcgaataatccacgctggttcaatctaaatacttataagactaaggaagagatgattgttgctacctcccagactagccagtacggcggtgatctaacaaatacattcggagcgatccagtatgcgcgaaaatatgcgtattcagcggcctctggaggccgtcgaagtgcaacacttaaagtaatggtg

SEQ ID NO: 8 - protein, 87 aa, Artificial Sequence
  Description of Artificial Sequence Integrin
NFLEKFVQGLDIGPTKTQVGLIQYANNPRWFNLNTYKTKEEMIVATSQTSQYGGDLTNTFGAIQYARKYAYSAASGGRRSATLKVMV

SEQ ID NO: 9 - DNA, 294 bp, Artificial Sequence
  Description of Artificial Sequence Integrin
tacaacgtcgacacagaatctgcacttttatatcagggcccgcataatacactgtttggctacagttggctccactcccatggagctcatagatggctactggtaggagcgccaacagcaatgtggttagcaatggcaagcgttattaatcctggggccatctatagatgcagaataggaaaaaacccagggcagacgtgtgaattgcaattgggttcattccacggtgagcccggcggtaagacttgtctagaggaaagagatcaccaatggcttggggtgaccctctcgaga

SEQ ID NO: 10 - protein, 98 aa, Artificial Sequence
  Description of Artificial Sequence Integrin
YNVDTESALLYQGPHNTLFGYSWLHSHGAHRWLLVGAPTAMWLAMASVINPGAIYRCRIGKNPGQTCELQLGSFHGEPGGKTCLEERDHQWLGVTLSR

SEQ ID NO: 11 - DNA, 156 bp, Artificial Sequence
  Description of Artificial Sequence Integrin
caggattatgtaaagaaattcggcgaacattttgcaagttgtcaagcagggatatcctcgttctatacgaaagacttaatcgtaatgggtgcaccaggatcttcatactggacaggaagcttatttgtatacatgattaccactaataagtataaa

SEQ ID NO: 12 - protein, 52 aa, Artificial Sequence
  Description of Artificial Sequence Integrin
QDYVKKFGEHFASCQAGISSFYTKDLIVMGAPGSSYWTGSLFVYMITTNKYK

SEQ ID NO: 13 - DNA, 156 bp, Artificial Sequence; identical to SEQ ID NO: 11
  Description of Artificial Sequence Integrin

SEQ ID NO: 14 - protein, 52 aa, Artificial Sequence; identical to SEQ ID NO: 12
  Description of Artificial Sequence Integrin

SEQ ID NO: 15 - DNA, 60 bp, Artificial Sequence
  Description of Artificial Sequence Integrin
ggacatagatggaaaaacatattttatataaagaatgaaaataaattaccaacaggagga

SEQ ID NO: 16 - protein, 20 aa, Artificial Sequence
  Description of Artificial Sequence Integrin
GHRWKNIFYIKNENKLPTGG

SEQ ID NO: 17 - DNA, 33 bp, Artificial Sequence
  Description of Artificial Sequence Integrin
ggaggagcaccacagcatgaacaaataggaaaa

SEQ ID NO: 18 - protein, 11 aa, Artificial Sequence
  Description of Artificial Sequence Integrin
GGAPQHEQIGK

SEQ ID NO: 19 - DNA, 18 bp, Artificial Sequence
  Description of Artificial Sequence Integrin
agttattggacaggaagt

SEQ ID NO: 20 - protein, 6 aa, Artificial Sequence
  Description of Artificial Sequence Integrin
SYWTGS

SEQ ID NO: 21 - DNA, 33 bp, Artificial Sequence
  Description of Artificial Sequence Integrin
atgggagcaccaggaagtagttattggacagga

SEQ ID NO: 22 - protein, 11 aa, Artificial Sequence
  Description of Artificial Sequence Integrin
MGAPGSSYWTG

SEQ ID NO: 23 - DNA, 111 bp, Artificial Sequence
  Description of Artificial Sequence Integrin
tacaatgtagatacagaaagtgcattactctatcaaggtccacacaacacattgtttgggtatagttggcttcatagtcatggagcacacagatggctgctagtaggcgca

SEQ ID NO: 24 - protein, 37 aa, Artificial Sequence
  Description of Artificial Sequence Integrin
YNVDTESALLYQGPHNTLFGYSWLHSHGAHRWLLVGA

SEQ ID NO: 25 - DNA, 225 bp, Artificial Sequence
  Description of Artificial Sequence Integrin
atagtaacgtgtggccatagatggaaaaatattttttatatcaaacacgaaaacaaattaccaacaggagggtgttatggcgtgcccccggatttaagaaccgaattaagtaagagaatagcccctggttatcaggactacgttaaaaagttcggagagcattttgctagttgccaagcaggtatcagtagtttctacactaaggatttaattgtcatgggggcg

SEQ ID NO: 26 - protein, 75 aa, Artificial Sequence
  Description of Artificial Sequence Integrin
IVTCGHRWKNIFYIKHENKLPTGGCYGVPPDLRTELSKRIAPGYQDYVKKFGEHFASCQAGISSFYTKDLIVMGA

SEQ ID NO: 27 - DNA, 222 bp, Artificial Sequence
  Description of Artificial Sequence Integrin
tacatgattaccactaacaagtataaagcgtttttagggaagcaaaatcaggtgaagccaggaagttatttagggtatagtgtaggtgccggccatttcagaagtcaacacacgacagaagttgtcggcggtgcaccacaacatgagcagataggaaaagcttacatctttagtatagatgaaaaagaattaaatatattacacgagatgaagggaaaaaaa

SEQ ID NO: 28 - protein, 74 aa, Artificial Sequence
  Description of Artificial Sequence Integrin
YMITTNKYKAFLGKQNQVKPGSYLGYSVGAGHFRSQHTTEVVGGAPQHEQIGKAYIFSIDEKELNILHEMKGKK

SEQ ID NO: 29 - DNA, 849 bp, Artificial Sequence
  Description of Artificial Sequence Integrin
ttaggatcatatttcggagcatccgtcggcgcagtcgacttacacgctgatggcttctcagacctgctcgtcggtgctcccatgcaatcgacgataagagaagagggtagagtttttgtttacatcaattctggaagcggggcagttatgaacgcaatggagacaaacttagtgggaagtgacaaatacgcagcgcgatttggggaatccatcgtgaatttgggagatattgacaatgacgggtttgaagacgtagcgattggagcaccacaggaggacgatctccagggagctatctatatctacaacggcagagcggatggtatatcttcaacattttcccaaagaattgagggcctacaaatatcgaagtcgctatccatgtttgggcagagtatttctggtcagatcgacgcggataacaatggctatgtggatgtagcagtaggcgcgttcaggagtgatcgtagcgattctgctgttttgttaagaacgcgtccagtcgtcatagtggacgcttcacttagtcatcctgaatcagtaaaccgaacaaagtttgattgtgtcgagaatgggtggccgagcgtgtgtatagatctgacattatgcttctcgtacaaagggaaggaagttcctggttatattgtattattctacaatatgagtcttgatgttaaccgcaaagccgaatcgccaccgcggttttatttcagtagcaatggtactagtgatgtaattactggaagcatacaagtgtcttccagagaagccaactgccggacccatcaagccttcatgcgcaaagacgtaagggacatattaacccccatacagatcgaggccgcctat

SEQ ID NO: 30 - protein, 283 aa, Artificial Sequence
  Description of Artificial Sequence Integrin
LGSYFGASVGAVDLHADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAY

SEQ ID NO: 31 - DNA, 1032 bp, Artificial Sequence
  Description of Artificial Sequence Integrin
tcctcaatatatgacgactcctacctcggatacagtgtagcggtcggcgaattttcgggagacgacacagaagattttgtagctggggtgcccaaagggaatttgacttatggctacgttaccatactaaatggttctgatattcgtagtttatataatttcagtggggagcaaatggcaagctatttcggatatgcggtagcagcgaccgacgtcaacggtgatgggctggacgatttgcttgtcggggccccgttacttatggaccgcactccagatggaagaccacaggaagtgggtcgtgtatatgtgtacttacagcacccagcaggtatagagccgacaccgactttgacgctaaccggacacgacgagttcggccggtttggcagttcattaacaccccttggagacttagatcaggatggatacaatgacgttgctattggggcaccatttggtggcgaaacgcaacaaggtgtagtattcgtgtttcctggaggccctggaggcttaggcagtaaaccttcgcaagttttgcagccactatgggccgctagccatacgcccgatttctttggcagcgctctgagaggggggagggacctcgacggtaacgggtatcctgatctgatcgttggtagttttggagtcgataaggcggtggtctacagaggggggcccatagtttcagcaagtgccagccttacgatattccccgccatgtttaatcctgaggagagatcttgctcattggaaggtaacccggtcgcgtgtatcaacctctccttctgtttaaacgcatcgggtaaacatgtggctgattcgatcggatttacagtagaacttcaactagattggcagaagcaaaaaggcggagttagacgagccctcttcctcgcatccaggcaggcgactttaacacaaaccctactgatacagaacggagccagagaggattgccgcgaaatgaagatctacctgagaaatgaatctgagttccgagacaagttatctccgattcatattgct

SEQ ID NO: 32 - protein, 344 aa, Artificial Sequence
  Description of Artificial Sequence Integrin
SSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGGPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIA

SEQ ID NO: 33 - DNA, 660 bp, Artificial Sequence
  Description of Artificial Sequence Integrin
agctacctaggatatagtgttgctgtaggcgagttcagcggagatgatacagaagactttgttgcaggggtgcctaaggggaatctaacatatgggtacgtaacaatcctcaacggatcggatattcgtagtttatacaatttctccggtgagcaaatggcctcatattttggatacgccgttgcggctacggacgttaacggtgacggattagacgatcttcttgtgggagctcccctgctgatggaccgaacccctgatggtagaccccaggaagtcggaagagtctacgtctacttgcaacatcccgccggcatagaaccaacgccaactttaactctcactgggcatgacgaatttggtagattcggttcctctttaacccctcttggcgacttggaccaggatggatataatgatgtggcaataggcgcgccgtttgggggggagacccagcaaggcgtggtgttcgtctttccaggtggaccgggtgggctagggtctaaaccatcacaagttttacagccattatgggcagcgagtcacacgccagattttttcggcagtgcactcaggggtggacgggacttggacggcaacggctatccggatctgatagtagggtcgttcggtgtagataaagcagtagtctatcgcggg

SEQ ID NO: 34 - protein, 220 aa, Artificial Sequence
  Description of Artificial Sequence Integrin
SYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRG

SEQ ID NO: 35 - DNA, 360 bp, Artificial Sequence
  Description of Artificial Sequence Integrin
gcacatggttcgagcatcttagcatgcgctcctctctacagctggagaacggaaaaagaacccttatctgatccggtcgggacgtgttatttatcgaccgacaactttacaagaatcttagagtacgcgccatgtagatctgatttcagttgggcagcgggtcaagggtattgccaaggcggcttcagtgccgaatttactaagaccggaagagtagtgcttggaggtccaggatcatacttttggcaggggcaaattctatccgctacacaagagcagatagcagagagttattatccagaatacctgataaatttagttcagggccagttgcagactagacaagcctcatccatttat

SEQ ID NO: 36 - protein, 120 aa, Artificial Sequence
  Description of Artificial Sequence Integrin
AHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIY

SEQ ID NO: 37 - DNA, 18 bp, Artificial Sequence
  Description of Artificial Sequence Integrin
gattttagttgggcagca

SEQ ID NO: 38 - protein, 6 aa, Artificial Sequence
  Description of Artificial Sequence Integrin
DFSWAA

SEQ ID NO: 39 - DNA, 72 bp, Artificial Sequence
  Description of Artificial Sequence Integrin
ggagtagacgtagatcaggatggcgaaacagagttaataggagcaccattattttatggtgaacaaagaggg

SEQ ID NO: 40 - protein, 24 aa, Artificial Sequence
  Description of Artificial Sequence Integrin
GVDVDQDGETELIGAPLFYGEQRG

SEQ ID NO: 41 - DNA, 72 bp, Artificial Sequence
  Description of Artificial Sequence Integrin
ataacagatggagaagcaacagacagtggacaaattgatgcagcaaaagacatcatatatattataggaatc

SEQ ID NO: 42 - protein, 24 aa, Artificial Sequence
  Description of Artificial Sequence Integrin
ITDGEATDSGQIDAAKDIIYIIGI

SEQ ID NO: 43 - DNA, 30 bp, Artificial Sequence
  Description of Artificial Sequence Integrin
ataacagatggagaagcaacaagtggatgt

SEQ ID NO: 44 - protein, 10 aa, Artificial Sequence
  Description of Artificial Sequence Integrin
ITDGEATSGC

SEQ ID NO: 45 - DNA, 33 bp, Artificial Sequence
  Description of Artificial Sequence Integrin
ggagtagacgtagatcaagatggagaaacatgt

SEQ ID NO: 46 - protein, 11 aa, Artificial Sequence
  Description of Artificial Sequence Integrin
GVDVDQDGETC

SEQ ID NO: 47 - DNA, 24 bp, Artificial Sequence
  Description of Artificial Sequence Integrin
tgcccaaataaggaaaaagagtgt

SEQ ID NO: 48 - protein, 8 aa, Artificial Sequence
  Description of Artificial Sequence Integrin
CPNKEKEC

SEQ ID NO: 49 - DNA, 18 bp, Artificial Sequence
  Description of Artificial Sequence Integrin
aaagaatttgtaagtaca

SEQ ID NO: 50 - protein, 6 aa, Artificial Sequence
  Description of Artificial Sequence Integrin
KEFVST

SEQ ID NO: 51 - DNA, 51 bp, Artificial Sequence
  Description of Artificial Sequence Integrin
ccaataacacaattattaggaagaacccatacggcaactggaataagaaaa

SEQ ID NO: 52 - protein, 17 aa, Artificial Sequence
  Description of Artificial Sequence Integrin
PITQLLGRTHTATGIRK

SEQ ID NO: 53 - DNA, 51 bp, Artificial Sequence
  Description of Artificial Sequence Integrin
aaatttggagacccattaggatatgaagatgtaataccagaggcagataga

SEQ ID NO: 54 - protein, 17 aa, Artificial Sequence
  Description of Artificial Sequence Integrin
KFGDPLGYEDVIPEADR

SEQ ID NO: 55 - DNA, 72 bp, Artificial Sequence
  Description of Artificial Sequence Integrin
ggatgtccacaagaagatagtgacattgcattcttaatagatggaagtggaagtataatcccacatgacttt

SEQ ID NO: 56 - protein, 24 aa, Artificial Sequence
  Description of Artificial Sequence Integrin
GCPQEDSDIAFLIDGSGSIIPHDF

SEQ ID NO: 57 - DNA, 69 bp, Artificial Sequence
  Description of Artificial Sequence Integrin
tttagaagaatgaaagagtttgtaagtacagtaatggaacaattaaagaaaagtaagacattattcagt

SEQ ID NO: 58 - protein, 23 aa, Artificial Sequence
  Description of Artificial Sequence Integrin
FRRMKEFVSTVMEQLKKSKTLFS

SEQ ID NO: 59 - DNA, 54 bp, Artificial Sequence
  Description of Artificial Sequence Integrin
ggaaatagttttccagcaagtttagtagtagcagcagaagagggagagagagaa

SEQ ID NO: 60 - protein, 18 aa, Artificial Sequence
  Description of Artificial Sequence Integrin
GNSFPASLVVAAEEGERE

SEQ ID NO: 61 - DNA, 84 bp, Artificial Sequence
  Description of Artificial Sequence Integrin
aacgcacaaatcggaattgcaatgttagtaagtgtaggaaatttagaggaagcaggagaaagtgtaagttttcaattacagata

SEQ ID NO: 62 - protein, 28 aa, Artificial Sequence
  Description of Artificial Sequence Integrin
NAQIGIAMLVSVGNLEEAGESVSFQLQI

SEQ ID NO: 63 - DNA, 54 bp, Artificial Sequence
  Description of Artificial Sequence Integrin
acattaggaccaagtcaagaagagacaggaggagtatttttatgtccatggaga

SEQ ID NO: 64 - protein, 18 aa, Artificial Sequence
  Description of Artificial Sequence Integrin
TLGPSQEETGGVFLCPWR

SEQ ID NO: 65 - DNA, 39 bp, Artificial Sequence
  Description of Artificial Sequence Integrin
gcagaaggaggacaatgtccaagtttattatttgattta

SEQ ID NO: 66 - protein, 13 aa, Artificial Sequence
  Description of Artificial Sequence Integrin
AEGGQCPSLLFDL

SEQ ID NO: 67 - DNA, 117 bp, Artificial Sequence
  Description of Artificial Sequence Integrin
gccatggtcacagtattggcatttctttggctcccaagtctatatcagagaccactggatcaatttgtgttacaaagtcatgcttggttcaatgttagtagtttaccatacgcggta

SEQ ID NO: 68 - protein, 39 aa, Artificial Sequence
  Description of Artificial Sequence Integrin
AMVTVLAFLWLPSLYQRPLDQFVLQSHAWFNVSSLPYAV

SEQ ID NO: 69 - DNA, 36 bp, Artificial Sequence
  Description of Artificial Sequence Integrin
ggagcacattatatgagagcattaagtaatgtagaa

SEQ ID NO: 70 - protein, 12 aa, Artificial Sequence
  Description of Artificial Sequence Integrin
GAHYMRALSNVE

SEQ ID NO: 71 - DNA, 12 bp, Artificial Sequence
  Description of Artificial Sequence Integrin
ggagcaccatta

SEQ ID NO: 72 - protein, 4 aa, Artificial Sequence
  Description of Artificial Sequence Integrin
GAPL

SEQ ID NO: 73 - DNA, 39 bp, Artificial Sequence
  Description of Artificial Sequence Integrin
ggagatggaagacatgacttattagtaggagcaccatta

SEQ ID NO: 74 - protein, 13 aa, Artificial Sequence
  Description of Artificial Sequence Integrin
GDGRHDLLVGAPL

SEQ ID NO: 75 - DNA, 33 bp, Artificial Sequence
  Description of Artificial Sequence Integrin
acagatgtaaatggagacggaagacatgattta

SEQ ID NO: 76 - protein, 11 aa, Artificial Sequence
  Description of Artificial Sequence Integrin
TDVNGDGRHDL

SEQ ID NO: 77 - DNA, 36 bp, Artificial Sequence
  Description of Artificial Sequence Integrin
ggagatggaagacatgacttattagtaggagcacca

SEQ ID NO: 78 - protein, 12 aa, Artificial Sequence
  Description of Artificial Sequence Integrin
GDGRHDLLVGAP

SEQ ID NO: 79 - DNA, 42 bp, Artificial Sequence
  Description of Artificial Sequence Integrin
ggagacggaagacatgatttattagtaggagcaccattatat

SEQ ID NO: 80 - protein, 14 aa, Artificial Sequence
  Description of Artificial Sequence Integrin
GDGRHDLLVGAPLY

SEQ ID NO: 81 - DNA, 681 bp, Artificial Sequence
  Description of Artificial Sequence Integrin
gaatttgacggtgatcttaatacgactgagtacgtcgtcggagcaccaacttggtcgtggacattaggcgcagtcgagatactcgacagttattatcagaggttacatagattacgtgcagaacagatggcgtcctactttggtcacagcgtagcggtaacggatgtgaacggagacggccgccatgacttgctagttggagctccgctctacatggagagtcgagcagatcgcaagcttgctgaagtgggccgagtatatcttttccttcaaccacggggtccccacgccctaggcgctcctagtttattgttaaccggaacacagttgtatggtagattcggatctgcaatagcgccactcggggatttggatagagatggctataacgatatagctgtggccgccccttacggaggaccctccggcagagggcaggttctggttttcctagggcaaagtgaagggttaaggtcaagaccgtctcaagtcttagactcgccatttccaaccggaagtgcgtttgggttcagtctccgtggtgcagtggacatcgatgacaatggttacccggatctaattgttggagcctacggggccaatcaagtagcagtatatcgggcgcagcccgtagttaaagcttcagtccaactgctgctgcaagacagcctgaaccctgca

SEQ ID NO: 82 - protein, 227 aa, Artificial Sequence
  Description of Artificial Sequence Integrin
EFDGDLNTTEYVVGAPTWSWTLGAVEILDSYYQRLHRLRAEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLLQDSLNPA

SEQ ID NO: 83 - DNA, 60 bp, Artificial Sequence
  Description of Artificial Sequence Integrin
gcagtaacagatgtaaatggagacggaagacatgatttattagtaggagcaccattatat

SEQ ID NO: 84 - protein, 20 aa, Artificial Sequence
  Description of Artificial Sequence Integrin
AVTDVNGDGRHDLLVGAPLY

SEQ ID NO: 85 - DNA, 882 bp, Artificial Sequence
  Description of Artificial Sequence Integrin
ttttcctcagtcgtgacacaagctggcgagttagtattgggggctcccggaggctactacttcctggggctactcgcacaggcacccgtggcggacatattctcgtcttatagacctgggattttgttgtggcacgtctcctctcagtctttaagtttcgatagtagcaatccagaatattttgacggatactgggggtattctgtggcagtcggtgagttcgatggtgatctgaatactacagaatatgtggtaggggctcctacatggagttggactttaggcgcggtcgagatattagatagctactaccaacgcttacacagattgcgtgctgaacaaatggcctcctactttggtcattcagtcgccgttaccgatgtgaatggtgatggacggcatgacctcctagttggagctccactttacatggagagcagagcggaccgaaagttagctgaagtaggaagagtttatttgttcctacaaccgaggggcccgcatgcgcttggcgcaccttccttacttctgaccggtacgcaactttacgggcgatttgggtcggccattgcgccactgggggaccttgatcgcgacggatataacgacatcgcagttgccgcgccttatggaggcccatcgggtcggggacaggttctagtgttcctcggtcaaagtgaaggcctccgtagtagaccgagccaggtactggacagtccgtttcccacgggctcggcttttggtttttcattaagaggtgcggtagacatcgatgataacggataccccgatctcatagtaggggcctatggcgccaaccaggtcgcagtttatagggcccagccagtagtgaaagcatcagtccaattactagttcaggac

SEQ ID NO: 86 - protein, 294 aa, Artificial Sequence
  Description of Artificial Sequence Integrin
FSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRAEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQD

SEQ ID NO: 87 - DNA, 21 bp, Artificial Sequence
  Description of Artificial Sequence Integrin
gtagaaaatgattttagttgg

SEQ ID NO: 88 - protein, 7 aa, Artificial Sequence
  Description of Artificial Sequence Integrin
VENDFSW

SEQ ID NO: 89 - DNA, 15 bp, Artificial Sequence
  Description of Artificial Sequence Integrin
ggagaattagtatta

SEQ ID NO: 90 - protein, 5 aa, Artificial Sequence
  Description of Artificial Sequence Integrin
GELVL

SEQ ID NO: 91 - DNA, 39 bp, Artificial Sequence
  Description of Artificial Sequence Integrin
gatttatattatttaatggacttaagttacagtatgaaa

SEQ ID NO: 92 - protein, 13 aa, Artificial Sequence
  Description of Artificial Sequence Integrin
DLYYLMDLSYSMK

SEQ ID NO: 93 - protein, 16 aa, Artificial
  Integrin
DXXXXXDXSXSXKDDL

SEQ ID NO: 94 - DNA, 324 bp, Artificial Sequence
  Description of Artificial Sequence Integrin
tactgccgaaaagaaaactcatcggaaatatgtagtaacaatggggagtgcgtctgcggccaatgtgtatgccggaaacgtgacaacacaaacgaaatctatagtggaaagttttgtgagtgtgataatttcaactgtgatcgcagcaatggcttaatatgcggtggcaatggagtttgcaagtgtagggtgtgtgaatgcaatccaaattatacagggagtgcatgcgattgctctttagacactagtacgtgcgaggcatccaacgggcagatatgtaatggaagaggtatttgtgagtgtggtgtatgcaaatgtaccgac

SEQ ID NO: 95 - protein, 108 aa, Artificial Sequence
  Description of Artificial Sequence Integrin
YCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTD

SEQ ID NO: 96 - DNA, 21 bp, Artificial Sequence
  Description of Artificial Sequence Integrin
tgtacaagtgaacaaaattgc

SEQ ID NO: 97 - protein, 7 aa, Artificial Sequence
  Description of Artificial Sequence Integrin
CTSEQNC

SEQ ID NO: 98 - DNA, 708 bp, Artificial Sequence
  Description of Artificial Sequence Integrin
ttacgattacgctcgggcgaaccccagacatttacgcttaagttcaaacgggctgaggattatcctatcgacctttactatcttatggatctctcatatagtatgaaagatgatctggagaatgttaagtccttagggaccgatttaatgaacgagatgagaagaatcacttcagacttcagaattggatttggctcttttgtcgaaaaaaccgtaatgccatacataagcacaaccccagcaaagctgaggaatccgtgtacatcggagcaaaactgcactactcccttcagttataagaatgttctcagtctgacgaacaaaggggaagtatttaacgagctagtgggaaaacagagaattagcggtaacctcgactctccagaaggtggttttgatgcaattatgcaagttgcagtgtgtggatctctaatagggtggcgtaatgtaactagactattggtgttttccaccgacgccggcttccacttcgctggagacggcaagctagggggaatcgtattgcctaacgatggtcagtgccatttggaaaataatatgtatacgatgtcgcactactacgactacccatccatagcccatttagtccaaaagctgagcgaaaacaatattcaaacaatatttgcggtaacggaagagttccagccagtctataaggagcttaaaaatctcatcccgaaatcagcg

SEQ ID NO: 99 - protein, 236 aa, Artificial Sequence
  Description of Artificial Sequence Integrin
LRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTTPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSA

SEQ ID NO: 100 - DNA, 36 bp, Artificial Sequence
  Description of Artificial Sequence Integrin
aacaagggagaagtatttaatgagttagtaggaaaa

SEQ ID NO: 101 - protein, 12 aa, Artificial Sequence
  Description of Artificial Sequence Integrin
NKGEVFNELVGK

SEQ ID NO: 102 - DNA, 15 bp, Artificial Sequence
  Description of Artificial Sequence Integrin
acagcagaaaaatta

SEQ ID NO: 103 - protein, 5 aa, Artificial Sequence
  Description of Artificial Sequence Integrin
TAEKL

SEQ ID NO: 104 - DNA, 78 bp, Artificial Sequence
  Description of Artificial Sequence Integrin
gattacccaatagacttatactatttaatggacttaagttatagtatgaaggatgatttagaagtaaaaagtttagga

SEQ ID NO: 105 - protein, 26 aa, Artificial Sequence
  Description of Artificial Sequence Integrin
DYPIDLYYLMDLSYSMKDDLEVKSLG

SEQ ID NO: 106 - DNA, 60 bp, Artificial Sequence
  Description of Artificial Sequence Integrin
aatgtaaagagtttaggaacagcattaatgagagagatggaaaaaataacaagtgatttt

SEQ ID NO: 107 - protein, 20 aa, Artificial Sequence
  Description of Artificial Sequence Integrin
NVKSLGTALMREMEKITSDF

SEQ ID NO: 108 - DNA, 744 bp, Artificial Sequence
  Description of Artificial Sequence Integrin
ggacaaaaacagttaagtccgcagaaggtcactctatacttgcgtcccgggcaagcagccgcgttcaacgtaacgtttcgtcgcgcaaaaggatacccaatagacctttattatttaatggatttatcctactcaatgctcgatgatttaagaaacgttaagaagttaggcggggatctgctcagagctctcaatgagataactgaaagtggtcggataggtttcggttcgttcgttgataagacggtgctgccctttgtaaatacacacccagacaaactgaggaacccctgcccaaataaggagaaagaatgccagccgcctttcgcttttcgccatgtcctaaaattaacaaataatagcaatcaatttcagaccgaggtaggaaaacaacttattagtggaaacttagacgccccagagggcggcttagacgcaatgatgcaagtagcagcctgtccggaggaaattggttggcggaatgtcaccaggttgttggtatttgccactgacgatggattccattttgctggagatggcaagctaggggcgattcttacccctaacgacgggcgatgtcacctcgaagacaacctatataagagaagtaatgaattcgattatccatctgtgggacaactggcgcataagttggctgagaacaacatacagccaatctttgcagttacaagtcgaatggtgaaaacatacgaaaaacttacggaaatcatccctaaaagtgcg

SEQ ID NO: 109 - protein, 248 aa, Artificial Sequence
  Description of Artificial Sequence Integrin
GQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSA

SEQ ID NO: 110 - DNA, 783 bp, Artificial Sequence
  Description of Artificial Sequence Integrin
tacccaatagatctctactacctgatggatctatcctattcaatgctggacgatctacgtaacgttaagaaacttggaggtgatttactaagagctcttaacgaaatcacggagagtgggcgaatcggcttcggctcattcgtcgacaagacagtattgcccttcgtaaacacgcacccagacaagcttagaaacccctgcccaaataaagagaaagagtgtcaacccccgtttgcctttagacatgtcttaaagctcacgaataacagcaatcagtttcagacagaagttggaaaacaactgatatcgggtaatctagacgcaccagaggggggacttgatgccatgatgcaggtggcagcctgcccggaggaaattgggtggaggaatgtcacaagactgctagttttcgcaactgatgacgggtttcattttgctggagatggtaaactgggcgcaattttgactcctaacgatggacggtgtcatttggaagacaacctctataaaagaagcaatgaattcgactatcctagtgtaggtcaattagcgcacaagttagcagaaaacaatatacaaccgatatttgcggttaccagtcgcatggtgaaaacatacgaaaagttaaccgagataattccaaaatctgctgtgggcgagctctccgaagatagtagtaatgtcgtacacttgatcaagaatgcatataacaaattatctagtagagtatttttggaccataatgcgcttcctgatactctcaaggtgacctatgactcgttc

SEQ ID NO: 111 - protein, 261 aa, Artificial Sequence
  Description of Artificial Sequence Integrin
YPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSF

SEQ ID NO: 112 - DNA, 15 bp, Artificial Sequence
  Description of Artificial Sequence Integrin
agaaatgtaaaaaag

SEQ ID NO: 113 - protein, 5 aa, Artificial Sequence
  Description of Artificial Sequence Integrin
RNVKK

SEQ ID NO: 114 - DNA, 15 bp, Artificial Sequence
  Description of Artificial Sequence Integrin
caaccaccatttgca

SEQ ID NO: 115 - protein, 5 aa, Artificial Sequence
  Description of Artificial Sequence Integrin
QPPFA

SEQ ID NO: 116 - DNA, 18 bp, Artificial Sequence
  Description of Artificial Sequence Integrin
ttaataagtggaaattta

SEQ ID NO: 117 - protein, 6 aa, Artificial Sequence
  Description of Artificial Sequence Integrin
LISGNL

SEQ ID NO: 118 - DNA, 15 bp, Artificial Sequence
  Description of Artificial Sequence Integrin
ggacaattagcacat

SEQ ID NO: 119 - protein, 5 aa, Artificial Sequence
  Description of Artificial Sequence Integrin
GQLAH

SEQ ID NO: 120 - DNA, 267 bp, Artificial Sequence
  Description of Artificial Sequence Integrin
gagctctcagaagattctagtaatgtcgtccatttaatcaaaaacgcctataacaaactaagttcgagagttttcttagaccacaatgcactgccagatacgttgaaggtaacatacgacagcttttgctccaatggggtgacccatagaaaccagccaagaggcgattgtgacggagtacaaataaatgtaccaataacattccaggttaaggtgacagctactgagtgtatacaagaacaaagttttgtaattagagcgcttggt

SEQ ID NO: 121 - protein, 89 aa, Artificial Sequence
  Description of Artificial Sequence Integrin
ELSEDSSNVVHLIKNAYNKLSSRVFLDHNALPDTLKVTYDSFCSNGVTHRNQPRGDCDGVQINVPITFQVKVTATECIQEQSFVIRALG

SEQ ID NO: 122 - DNA, 168 bp, Artificial Sequence
  Description of Artificial Sequence Integrin
ggtttcaccgacattgtaacagtacaggtattaccacaatgcgaatgcagatgtagagatcaaagtagagacagaagtttatgccatggaaagggctttttagaatgtggaatctgtagatgcgatacgggatatataggaaaaaattgtgagtgtcagactcaaggg

SEQ ID NO: 123 - protein, 56 aa, Artificial Sequence
  Description of Artificial Sequence Integrin
GFTDIVTVQVLPQCECRCRDQSRDRSLCHGKGFLECGICRCDTGYIGKNCECQTQG

SEQ ID NO: 124 - DNA, 45 bp, Artificial Sequence
  Description of Artificial Sequence Integrin
tgtaatgcatttaagatattagtagtaataacagatggagaaaaa

SEQ ID NO: 125 - protein, 15 aa, Artificial Sequence
  Description of Artificial Sequence Integrin
CNAFKILVVITDGEK

SEQ ID NO: 126 - DNA, 60 bp, Artificial Sequence
  Description of Artificial Sequence Integrin
acaggaataagaaaggtagtaagagaattatttaatataacaaacggagcaagaaaaaat

SEQ ID NO: 127 - protein, 20 aa, Artificial Sequence
  Description of Artificial Sequence Integrin
TGIRKVVRELFNITNGARKN

SEQ ID NO: 128 - DNA, 75 bp, Artificial Sequence
  Description of Artificial Sequence Integrin
gatttaagttatagtctcgacgatctgagaaatgtaaagaaacttggaggagacctattaagagcattgaacgaa

SEQ ID NO: 129 - protein, 25 aa, Artificial Sequence
  Description of Artificial Sequence Integrin
DLSYSLDDLRNVKKLGGDLLRALNE

SEQ ID NO: 130 - DNA, 189 bp, Artificial Sequence
  Description of Artificial Sequence Integrin
gactatcccgtagacatatactaccttatggatttaagttactccatgaaggacgatctctggtcaattcagaacttgggaacaaaactagcaacacaaatgagaaagctgacatcgaatttaagaataggatttggagcattcgtagataaaccagtaagcccttatatgtatatctctccaccggaa

SEQ ID NO: 131 - protein, 63 aa, Artificial Sequence
  Description of Artificial Sequence Integrin
DYPVDIYYLMDLSYSMKDDLWSIQNLGTKLATQMRKLTSNLRIGFGAFVDKPVSPYMYISPPE

SEQ ID NO: 132 - DNA, 45 bp, Artificial Sequence
  Description of Artificial Sequence Integrin
gacgcaccagaaggaggatttgatgcaataatgcaagcaacagta

SEQ ID NO: 133 - protein, 15 aa, Artificial Sequence
  Description of Artificial Sequence Integrin
DAPEGGFDAIMQATV

SEQ ID NO: 134 - DNA, 363 bp, Artificial Sequence
  Description of Artificial Sequence Integrin
ttttccatacaggttcgacaggtagaggattatccagtagacatctattacttaatggacttaagctatagtatgaaggacgatctctggagtatacaaaatttaggtaccaagttggccacccaaatgcgtaaattaacttcaaatttacggataggattcggggcatttgtggataaacccgtatcgccgtacatgtatattagtccacctgaggcgcttgaaaacccctgctacgacatgaaaacaacgtgtctgcctatgtttggctacaagcatgtcctaacattaacggatcaagtcactaggttcaacgaggaagttaaaaagcagagtgtgtctcgcaatagagatgctccggaa

SEQ ID NO: 135 - protein, 121 aa, Artificial Sequence
  Description of Artificial Sequence Integrin
FSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPE

SEQ ID NO: 136 - DNA, 87 bp, Artificial Sequence
  Description of Artificial Sequence Integrin
ggagtaagtagttgccagcaatgtttagcagtaagtccaatgtgtgcatggtgcagtgatgaagcattaccattaggaagtccaaga

SEQ ID NO: 137 - protein, 29 aa, Artificial Sequence
  Description of Artificial Sequence Integrin
GVSSCQQCLAVSPMCAWCSDEALPLGSPR

SEQ ID NO: 138 - DNA, 63 bp, Artificial Sequence
  Description of Artificial Sequence Integrin
gtattagaagacagaccattaagtgataaaggaagtggagatagtagtcaagtaacacaggta

SEQ ID NO: 139 - protein, 21 aa, Artificial Sequence
  Description of Artificial Sequence Integrin
VLEDRPLSDKGSGDSSQVTQV

SEQ ID NO: 140 - DNA, 153 bp, Artificial Sequence
  Description of Artificial Sequence Integrin
aacatcaatttaatatttgcagtcacagaaaacgtagtgaatctttaccagaactatagtgagctaataccaggaacaacagtaggagttctcagtatggatagtagtaatgtactgcaattgattgtagacgcatatggaaaaataagaagt

SEQ ID NO: 141 - protein, 51 aa, Artificial Sequence
  Description of Artificial Sequence Integrin
NINLIFAVTENVVNLYQNYSELIPGTTVGVLSMDSSNVLQLIVDAYGKIRS

SEQ ID NO: 142 - DNA, 123 bp, Artificial Sequence
  Description of Artificial Sequence Integrin
ataggatttggagcattcgtagacaaaccagtaagtccttacatgtatataagtccacccgaagcattagagaatccatgctacgatatgaagacaacatgtttaccgatgtttggatataaa

SEQ ID NO: 143 - protein, 41 aa, Artificial Sequence
  Description of Artificial Sequence Integrin
IGFGAFVDKPVSPYMYISPPEALENPCYDMKTTCLPMFGYK

SEQ ID NO: 144 - DNA, 36 bp, Artificial Sequence
  Description of Artificial Sequence Integrin
agtgtaagtagaaatagagatgcaccagaaggagga

SEQ ID NO: 145 - protein, 12 aa, Artificial Sequence
  Description of Artificial Sequence Integrin
SVSRNRDAPEGG

SEQ ID NO: 146 - DNA, 33 bp, Artificial Sequence
  Description of Artificial Sequence Integrin
agtgatagtagaaatagagatgcaccagaagga

SEQ ID NO: 147 - protein, 11 aa, Artificial Sequence
  Description of Artificial Sequence Integrin
SDSRNRDAPEG

SEQ ID NO: 148 - DNA, 15 bp, Artificial Sequence
  Description of Artificial Sequence Integrin
agaaatagagatgca

SEQ ID NO: 149 - protein, 5 aa, Artificial Sequence
  Description of Artificial Sequence Integrin
RNRDA

SEQ ID NO: 150 - DNA, 45 bp, Artificial Sequence
  Description of Artificial Sequence Integrin
gatgcaccagaaggaggatttgacgcaataatgcaagcaacagta

SEQ ID NO: 151 - protein, 15 aa, Artificial Sequence
  Description of Artificial Sequence Integrin
DAPEGGFDAIMQATV

SEQ ID NO: 152 - DNA, 45 bp, Artificial Sequence; identical to SEQ ID NO: 150
  Description of Artificial Sequence Integrin

SEQ ID NO: 153 - protein, 15 aa, Artificial Sequence
  Description of Artificial Sequence Integrin
DAPEGGFDAIMQATV

SEQ ID NO: 154 - DNA, 258 bp, Artificial Sequence
  Description of Artificial Sequence Integrin
gatgcgccagaaggtgggtttgacgcgatcatgcaagctacagtgtgcgacgaaaaaataggctggagaaacgatgcaagtcacctccttgtcttcacaaccgatgcaaaaacacatattgccctggacgggagattggccggcatagttcaaccaaatgatggtcagtgtcatgtaggatcagacaatcactattctgctagcactacgatggattacccatccttaggattaatgacagagaagctatcgcagaag

SEQ ID NO: 155 - protein, 86 aa, Artificial Sequence
  Description of Artificial Sequence Integrin
DAPEGGFDAIMQATVCDEKIGWRNDASHLLVFTTDAKTHIALDGRLAGIVQPNDGQCHVGSDNHYSASTTMDYPSLGLMTEKLSQK

SEQ ID NO: 156 - DNA, 42 bp, Artificial Sequence
  Description of Artificial Sequence Integrin
atggacttaagttatagtatgaaagatgatttatggagtata

SEQ ID NO: 157 - protein, 14 aa, Artificial Sequence
  Description of Artificial Sequence Integrin
MDLSYSMKDDLWSI

SEQ ID NO: 158 - DNA, 18 bp, Artificial Sequence
  Description of Artificial Sequence Integrin
ggaccaaatatatgtaca

SEQ ID NO: 159 - protein, 6 aa, Artificial Sequence
  Description of Artificial Sequence Integrin
GPNICT

SEQ ID NO: 160 - DNA, 39 bp, Artificial Sequence
  Description of Artificial Sequence Integrin
ggaccaaatatatgtacaacaagaggagtaagtagttgc

SEQ ID NO: 161 - protein, 13 aa, Artificial Sequence
  Description of Artificial Sequence Integrin
GPNICTTRGVSSC

SEQ ID NO: 162 - DNA, 207 bp, Artificial Sequence
  Description of Artificial Sequence Integrin
aaagattctttaatagtacaggtaacatttgactgtgactgtgcatgtcaggcacaagcagaacccaactcgcatagatgcaacaatggaaatggcacattcgaatgcggagtatgcagatgcggaccgggttggttagggagtcagtgtgaatgctcagaggaagattatagaccttcccaacaagatgagtgtagcccaagagag

SEQ ID NO: 163 - protein, 69 aa, Artificial Sequence
  Description of Artificial Sequence Integrin
KDSLIVQVTFDCDCACQAQAEPNSHRCNNGNGTFECGVCRCGPGWLGSQCECSEEDYRPSQQDECSPRE

SEQ ID NO: 164 - DNA, 267 bp, Artificial Sequence
  Integrin
cctacttgcccggatgcttgcacttttaaaaaagaatgtgtagaatgcaaaaaatttgaccgtgagccctatatgacagaaaatacttgcaacaggtattgtagagatgaaatagagagcgttaaagagttaaaagatacaggtaaagatgcagttaactgtacatataaaaatgaggacgattgtgtggtacgattccaatattatgaagacagttcaggaaaatctatattgtatgtagtggaagagccagaatgtccaaaaggg

SEQ ID NO: 165 - protein, 89 aa, Artificial Sequence
  Integrin
PTCPDACTFKKECVECKKFDREPYMTENTCNRYCRDEIESVKELKDTGKDAVNCTYKNEDDCVVRFQYYEDSSGKSILYVVEEPECPKG

SEQ ID NO: 166 - DNA, 15 bp, Artificial Sequence
  Description of Artificial Sequence Integrin
aaagatgacttatgg

SEQ ID NO: 167 - protein, 5 aa, Artificial Sequence
  Description of Artificial Sequence Integrin
KDDLW

SEQ ID NO: 168 - DNA, 39 bp, Artificial Sequence
  Description of Artificial Sequence Integrin
agtgtaagtagaaatagagatgcaccagaaggaggattt

SEQ ID NO: 169 - protein, 13 aa, Artificial Sequence
  Description of Artificial Sequence Integrin
SVSRNRDAPEGGF

SEQ ID NO: 170 - DNA, 270 bp, Artificial Sequence
  Description of Artificial Sequence Integrin
cacgtggggagtgacaaccattattccgcatctacaactatggactatccaagtctgggcttaatgacagagaagttaagccaaaagaatttaaacttgatctttgcagttacagagaacgtagtcaatctttaccagaattacagtgagctaattccaggaacgaccgtaggagtattgtcgatggatagttcaaatgtcctccaactaatagtggatgcatatggtaaaataagaagtaaagttgaattagaagtaagagatctccca

SEQ ID NO: 171 - protein, 90 aa, Artificial Sequence
  Description of Artificial Sequence Integrin
HVGSDNHYSASTTMDYPSLGLMTEKLSQKNLNLIFAVTENVVNLYQNYSELIPGTTVGVLSMDSSNVLQLIVDAYGKIRSKVELEVRDLP

SEQ ID NO: 172 - DNA, 417 bp, Artificial Sequence
  Description of Artificial Sequence Integrin
gacgatagtaaaaatttcagtattcaagtacgacaagtagaagactatcccgttgacatctactatctaatggatttaagttacagtatgaaagatgatttatggagtatacagaatttggggaccaagcttgcaacccaaatgagaaagctgacatcgaacttaaggattggatttggagcattcgttgataagcctgtgtcaccgtatatgtacatctctcccccagaggctttagaaaatccgtgttacgacatgaaaacgacatgtttacctatgtttggttataaacatgtattaacgctcactgaccaggtaacacgttttaacgaagaggtcaagaaacagagcgtgtcccggaaccgcgatgcgccagagggcggattcgacgccataatgcaagcaactgtctgcgat

SEQ ID NO: 173 - protein, 139 aa, Artificial Sequence
  Description of Artificial Sequence Integrin
DDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCD

SEQ ID NO: 174 - DNA, 117 bp, Artificial Sequence
  Description of Artificial Sequence Integrin
tatatgtacataagtcccccggaagcattagagaatccttgttacgatatgaaaactacctgcttaccaatgtttggatataagcatgtattaacattaacggaccaagtaacaaga

SEQ ID NO: 175 - protein, 39 aa, Artificial Sequence
  Description of Artificial Sequence Integrin
YMYISPPEALENPCYDMKTTCLPMFGYKHVLTLTDQVTR

SEQ ID NO: 176 - DNA, 18 bp, Artificial Sequence
  Description of Artificial Sequence Integrin
agaaatagagatgcatat

SEQ ID NO: 177 - protein, 6 aa, Artificial Sequence
  Description of Artificial Sequence Integrin
RNRDAY

SEQ ID NO: 178 - DNA, 48 bp, Artificial Sequence
  Description of Artificial Sequence Integrin
gacgcaccagaaggaggatttgatgcaataatgcaagcaacagtatat

SEQ ID NO: 179 - protein, 16 aa, Artificial Sequence
  Description of Artificial Sequence Integrin
DAPEGGFDAIMQATVY

SEQ ID NO: 180 - DNA, 24 bp, Artificial Sequence
  Description of Artificial Sequence Integrin
tgctatgatatgaaaacaacatgt

SEQ ID NO: 181 - protein, 8 aa, Artificial Sequence
  Description of Artificial Sequence Integrin
CYDMKTTC

SEQ ID NO: 182 - DNA, 60 bp, Artificial Sequence
  Description of Artificial Sequence Integrin
aattttagtatacaggtaagacaagtagaagactatccagtagatatatattacttaatg

SEQ ID NO: 183 - protein, 20 aa, Artificial Sequence
  Description of Artificial Sequence Integrin
NFSIQVRQVEDYPVDIYYLM

SEQ ID NO: 184 - DNA, 15 bp, Artificial Sequence
  Description of Artificial Sequence Integrin
gatatgaaaacaaca

SEQ ID NO: 185 - protein, 5 aa, Artificial Sequence
  Description of Artificial Sequence Integrin
DMKTT

SEQ ID NO: 186 - DNA, 15 bp, Artificial Sequence
  Description of Artificial Sequence Integrin
ataagtccaccagca

SEQ ID NO: 187 - protein, 5 aa, Artificial Sequence
  Description of Artificial Sequence Integrin
ISPPA

SEQ ID NO: 188 - DNA, 36 bp, Artificial Sequence
  Description of Artificial Sequence Integrin
aaacaaagtgtaagtagaaatagagatgcaccagaa

SEQ ID NO: 189 - protein, 12 aa, Artificial Sequence
  Description of Artificial Sequence Integrin
KQSVSRNRDAPE

SEQ ID NO: 190 - DNA, 837 bp, Artificial Sequence
  Description of Artificial Sequence Integrin
gatgacagtaaaaattttagtatccaggtaagacaggtagaagattatccagtcgacatatattacctcatggacctgagttacagtatgaaggatgatctctggtcaattcaaaatctagggactaagcttgcgacgcaaatgagaaaattgacaagcaatttacgaattggatttggagcattcgtcgataagcctgttagtccttacatgtacatctcaccccctgaagccttagagaacccctgctatgacatgaaaaccacatgtttaccgatgtttggttataaacatgtgctcacgcttacggaccaagtgactcggttcaatgaggaagtaaaaaagcagtctgtcagtaggaaccgtgatgcaccggaaggaggatttgacgcgataatgcaagccacagtatgtgacgagaaaataggctggcgcaacgatgcatcccatttactggtgttcaccactgatgcgaaaacacacatcgcattggatggtagattggctggaatagtacagccaaatgatggccaatgccatgtcgggagcgacaaccactattcggcaagtaccacgatggactaccccagcttaggtctaatgactgagaagttatcgcagaagaaccttaacctaatcttcgctgtaacagaaaatgtagttaatttatatcaaaactactcggaactgataccgggaacaacagttggggtcttgtccatggactcaagtaatgttttacagctaattgtggacgcttatggcaagattagatccaaagtggagttagaagttagagatcttccagaggagctctctctgtcttttaacgccacc

SEQ ID NO: 191 - protein, 279 aa, Artificial Sequence
  Description of Artificial Sequence Integrin
DDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNLNLIFAVTENVVNLYQNYSELIPGTTVGVLSMDSSNVLQLIVDAYGKIRSKVELEVRDLPEELSLSFNAT

SEQ ID NO: 192 - DNA, 621 bp, Artificial Sequence
  Description of Artificial Sequence Integrin
gatgattctaagaatttttccatccaggttcgacaggtcgaagattacccagtagacatatattacctaatggatctcagttatagtatgaaggacgatctatggagtatccaaaacctgggcacgaaacttgccactcaaatgcggaaattaacatcaaacttgaggattggctttggggcattcgtggataaacccgtatccccatatatgtacatctctccaccggaggcactcgaaaacccttgctacgacatgaagaccacatgccttcctatgtttgggtataaacacgtgcttactttaaccgaccaggttacgagattcaatgaagaggtaaaaaagcaaagtgtaagccgtaacagagacgcaccggagggagggttcgacgcaataatgcaagctactgtctgtgacgagaagattggatggagaaatgatgcgtcgcatttgttagtctttacaacagatgccaaaacacacattgcgctggacggtcgcctcgcaggcatagttcagccaaatgatggtcagtgtcatgtgggtagtgataatcattatagcgcttcaacaaccatggactaccccagtctaggactgatgacggaaaagttgtcgcaa

SEQ ID NO: 193 - protein, 207 aa, Artificial Sequence
  Description of Artificial Sequence Integrin
DDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQ

SEQ ID NO: 194 - DNA, 1053 bp, Artificial Sequence
  Description of Artificial Sequence Integrin
aagcaactgaatttcacggcctctggagaggcagaggcccgcagatgcgcacggagggaagagctcctagctaggggatgccccctggaggagctagaagagccacgtggacagcaagaggtactacaggatcagccgctgtcgcaaggagcccgaggtgagggtgcgacccagctagcaccacaacgcgtacgcgttacattacggccaggcgaaccacaacaattacaggtaagatttttgcgtgctgaagggtatccggtggatttatactatctcatggatcttagttactccatgaaggatgatctagaaagggtacgccaactgggtcatgccttattggtaagattacaagaagtaacacatagcgtacgtatcgggtttggatctttcgtagacaaaaccgttttacctttcgtgagtaccgtgcctagcaaattgcgtcacccttgtccaactaggcttgagcgatgccagagtccgttctcattccaccatgttttgagtttaactggagatgcccaggccttcgagcgagaagtcggccggcaatccgtttctgggaatttagacagtcccgagggagggtttgacgcgatacttcaagcagcgctctgtcaggaacagattggctggcgaaacgtcagcagactattagtctttacgagtgacgatacttttcacacagcaggggacggaaagcttggcggtatttttatgcccagcgacggtcattgtcacctcgattcaaatggattgtacagtcggtccacagaattcgattatccttcggtgggccaggtggcgcaggcactgagtgctgcaaacatccagccaatatttgctgttacatcggcggcgttgccggtttaccaagaactctcaaaattaatacccaaatccgctgtcggcgaattatctgaggactcctcaaacgtggtccaactcatcatggacgcttataattcgcttagtagcacggtaacactggaacactcatcgcttccgcccggtgtccatatttcttatgagagtcaatgtgaagggcct

SEQ ID NO: 195 - protein, 351 aa, Artificial Sequence
  Description of Artificial Sequence Integrin
KQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGP

SEQ ID NO: 196 - DNA, 273 bp, Artificial Sequence
  Description of Artificial Sequence Integrin
agttttgttgataaaacagtcctgccgttcgtaagtaccgtaccaagtaagttacgccatccatgtccaacgaggttggagagatgccagtctcctttttccttccaccatgtcttaagcctaactggtgacgctcaagcctttgaacgggaagtaggaagacaatcggtgagtgggaaccttgattcacccgaaggaggcttcgacgcaatattacaggcggcactctgtcaggagcaaataggatggcgaaatgttagtcgtttattagtg

SEQ ID NO: 197 - protein, 91 aa, Artificial Sequence
  Description of Artificial Sequence Integrin
SFVDKTVLPFVSTVPSKLRHPCPTRLERCQSPFSFHHVLSLTGDAQAFEREVGRQSVSGNLDSPEGGFDAILQAALCQEQIGWRNVSRLLV

SEQ ID NO: 198 - DNA, 312 bp, Artificial Sequence
  Description of Artificial Sequence Integrin
aaacaactcaatttcacagctagtggcgaagcagaggctaggagatgcgccaggcgagaagaattattggcacgcgggtgtcccctggaggagcttgaagagccacggggtcagcaggaagttttacaagatcaaccattaagtcagggagcacgcggcgaaggggcgacacaattagcgccacagcgtgtcagagtgacattgcgaccaggagagcctcaacagttacaagtacgttttcttcgggccgagggttacccggtagatctgtactacctaatggacctcagttatagtatgaaggacgatcta

SEQ ID NO: 199 - protein, 104 aa, Artificial Sequence
  Description of Artificial Sequence Integrin
KQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDL

SEQ ID NO: 200 - DNA, 1017 bp, Artificial Sequence
  Description of Artificial Sequence Integrin
gaaaaacgtgagggaaaagccgaagacagaggccagtgtaaccacgtgaggataaaccaaaccgtaaccttctgggtctcgcttcaggcaactcattgtttacccgaaccacatttgctacgcctccgggctttagggttttctgaggagctcatagttgagctacacacgttatgtgactgcaattgctcagacacgcaaccacaagcgccacactgttccgatgggcaggggcaccttcaatgtggagtctgtagttgcgctcctggtagattgggtaggctgtgcgagtgcagtgtagctgagttatcgagtcctgatctcgaaagcggatgtcgcgcgccgaatgggactggacctctgtgttccggaaaagggcattgccagtgtggtcggtgctcttgctcgggtcagtcaagtggccatttgtgcgaatgtgacgacgccagctgtgaacggcatgagggcattttgtgcgggggtttcggcaggtgccagtgtggggtgtgtcactgtcatgcaaaccgaacaggtcgagcatgcgagtgttccggcgacatggattcttgtataagtccggagggaggtttatgcagtggtcatggaagatgcaagtgcaatcgctgccaatgcttagatggttactacggcgccctatgtgatcagtgcccaggctgtaagactccatgtgaaagacaccgagactgcgcagagtgcggtgcgtttagaacaggccccctggccaccaattgcagcacagcttgtgctcacactaatgtgacgcttgcacttgcgcccatattagatgacggctggtgtaaagaaagaacattggataaccaactgttttttttcctagtagaagacgatgccagaggcacggtagttctccgtgttagaccgcaagaaaagggagcagatcatacccaagcaattgtactggggtgtgttgggggaatcgtcgcagtggggctagggctcgtacttgcgtatcgtttatcagtcgaaatctatgat

SEQ ID NO: 201 - protein, 339 aa, Artificial Sequence
  Description of Artificial Sequence Integrin
EKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYD

SEQ ID NO: 202 - DNA, 15 bp, Artificial Sequence
  Description of Artificial Sequence Integrin
gaacatataccagca

SEQ ID NO: 203 - protein, 5 aa, Artificial Sequence
  Description of Artificial Sequence Integrin
EHIPA

SEQ ID NO: 204 - DNA, 60 bp, Artificial Sequence
  Description of Artificial Sequence Integrin
ataccatgtaataacaaaggagcacatagtgtaggattaatgtggtggatgttagcaaga

SEQ ID NO: 205 - protein, 20 aa, Artificial Sequence
  Description of Artificial Sequence Integrin
IPCNNKGAHSVGLMWWMLAR

SEQ ID NO: 206 - DNA, 39 bp, Artificial Sequence
  Description of Artificial Sequence Integrin
aaagtaatattagatagaggaagtgtattagtaacatgt

SEQ ID NO: 207 - protein, 13 aa, Artificial Sequence
  Description of Artificial Sequence Integrin
KVILDRGSVLVTC

SEQ ID NO: 208 - DNA, 24 bp, Artificial Sequence
  Description of Artificial Sequence Integrin
tgctgggacgatggatggttatgt

SEQ ID NO: 209 - protein, 8 aa, Artificial Sequence
  Description of Artificial Sequence Integrin
CWDDGWLC

SEQ ID NO: 210 - DNA, 24 bp, Artificial Sequence
  Description of Artificial Sequence Integrin
tgctgggatgacttatggttatgt

SEQ ID NO: 211 - protein, 8 aa, Artificial Sequence
  Description of Artificial Sequence Integrin
CWDDLWLC

SEQ ID NO: 212 - DNA, 27 bp, Artificial Sequence
  Description of Artificial Sequence Integrin
tgcttattaagaatgagaagtatatgt

SEQ ID NO: 213 - protein, 9 aa, Artificial Sequence
  Description of Artificial Sequence Integrin
CLLRMRSIC

SEQ ID NO: 214 - DNA, 60 bp, Artificial Sequence
  Description of Artificial Sequence Integrin
ccagatacaagacccgcccctggaagtacagcaccgccagcgcatggagtaacaagtgct

SEQ ID NO: 215 - protein, 20 aa, Artificial Sequence
  Description of Artificial Sequence Integrin
PDTRPAPGSTAPPAHGVTSA

SEQ ID NO: 216 - DNA, 42 bp, Artificial Sequence
  Description of Artificial Sequence Integrin
gagtggtgtgaatatttaggaggatatttaagatgctacgca

SEQ ID NO: 217 - protein, 14 aa, Artificial Sequence
  Description of Artificial Sequence Integrin
EWCEYLGGYLRCYA

SEQ ID NO: 218 - DNA, 18 bp, Artificial Sequence
  Description of Artificial Sequence Integrin
gaatggccagagtattta

SEQ ID NO: 219 - protein, 6 aa, Artificial Sequence
  Description of Artificial Sequence Integrin
EWPEYL

(19) United States
(12) Patent Application Publication
(10) Pub. No.: US 2004/0127416 A1
(43) Pub. Date: Jul. 1, 2004

(54) Therapeutic bioconjugates

(76) Inventors: Stephen P. Massia, Mesa, AZ (US); Gholam Reza Ehteshami, Scottsdale, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/716,293

(22) Filed: Nov. 17, 2003

Related U.S. Application Data
(63) Continuation-in-part of application No. 10/295,734, filed on Nov. 15, 2002.

(51) Int. Cl. A61K 38/17; C07K 14/705

(52) U.S. Cl. 514/12; 530/350

(57) ABSTRACT

A therapeutic bioconjugate is composed of a hydrophilic polymer covalently bound to one or more peptides capable of binding specifically to a ligand expressed on a cell surface and thereby forming a biofilm to prevent attachment of cells with the binding partner of the ligand.

### CROSS REFERENCE

This application is a continuation in part of pending U.S. Utility application Ser. No. 10/295,734, filed Nov. 15, 2002, the disclosure of which is hereby incorporated by reference in its entirety.

### FIELD OF THE INVENTION

The present invention relates generally to biomaterials and, more specifically, to therapeutic conjugates of polymers and peptides capable of binding selectively to ligands expressed on certain cells in target tissues.

### SEQUENCE LISTING

This application also includes a Sequence Listing (158 pages) on paper and on one diskette and two Addenda, all of which are hereby incorporated by reference.

### BACKGROUND

Integrins are cell-bound molecules that aid cell-to-cell interactions by providing binding sites for other cells. The integrins are receptors that recognize specific ligands in a variety of physiological and pathological processes. Cellular interactions mediated by the integrins include adhesion, migration, release of soluble factors (cytokines, free radical species, degradative enzymes, etc.), and extracellular matrix (ECM) deposition. These cellular interactions affect pathological processes by reversing them or by sustaining, enhancing or amplifying them.

The integrin superfamily is an important and well characterized group of cell-surface receptors for both cell-substrate and cell-cell adhesion. Integrins are characteristically membrane-spanning heterodimeric protein complexes consisting of a α subunit and a β subunit. Eighteen distinct α subunits and eight distinct β subunits have currently been isolated and identified. While 144 combinations are theoretically possible, 24 αβ combinations have been observed. Integrin complexes containing the β₁ and β₃ subunits generally are involved in cell adhesion to the extracellular matrix, while the β₂ integrins are involved in cell-cell adhesion. The complement of integrins expressed by different cell types varies greatly. Depending on the cell type, mammalian cells express from two to ten different integrins, which are the means by which the cell senses its local environment and responds to changes in extracellular matrix composition and topography. Integrins were initially identified as cell-surface adhesion receptors mechanically linking the cell's cytoskeleton to the extracellular matrix or to other cells. Now integrins are also recognized as cell signaling receptors implicated in the regulation of cellular adhesion, migration, tumor metastasis, proliferation, angiogenesis, bone resorption, apoptosis, and gene expression.

The pivotal importance of integrins in health and disease has lead to a search for therapeutic strategies that target specific receptor-ligand interactions. Research efforts have generally focused on developing antibodies, peptides, and small molecules as therapeutic agents that selectively inhibit these specific receptor/ligand interactions and suppress pathological immune responses. Strategies for pharmacological modulation include blockade of receptors (the application of mAb, soluble ligands, and synthetic ligands); inhibition of expression of adhesion receptors (immunosuppressive and anti-inflammatory drugs, phosphodiesterase and proteosome inhibitors, antisense oligonucleotides); and inhibition of activation of integrins (antagonists of chemokines; anti-inflammatory drugs).

A threatening pathological condition involving specific receptor-ligand interactions is an excessive inflammatory response. Receptor-ligand interactions are critical for every step of an inflammatory response including neutrophil, monocyte, lymphocyte, and macrophage adhesion to vascular endothelial cells, transvascular migration into inflamed tissues, and phagocytosis of foreign bodies, injured tissues, pathogens, etc. During the inflammatory response, cell signaling releases degradative enzymes and oxidative free radicals to facilitate pathogen and injured tissue removal. Excessive inflammatory response results in the release of these degradative agents at abnormally high levels, damaging healthy tissue.

One therapeutic approach involves antibodies that are effective in immunomodulation. Researchers have evaluated the effects of post-injury treatment with antibody inhibitors of CD11b/CD18 on pathogenic immune responses. Post-injury treatment with monoclonal antibodies directed against CD11b (integrin α_{M} subunit) has reduced intestinal ischemia/reperfusion-mediated lung and liver injury without affecting levels of circulating and sequestered PMNs. Monoclonal antibody directed against CD18 (integrin β₂ subunit) has effectively reduced intestinal ischemia/reperfusion-mediated tissue injury in vivo. Preclinical studies have also shown that anti-ICAM-1 and anti-CD11b/CD18 therapies can increase tolerance (decrease rejection) in several transplantation models including cardiac, cornea, skin, pancreatic islet, and peripheral nerve allografts.

In another approach, antisense oligonucleotides, blocking ICAM-1 expression in donor and host tissues, are being developed to limit reperfusion injury and decrease allograft rejection rates for heart and kidney transplant.

However, the current therapeutic regimens against CD11b/CD 18 are limited to local delivery because systemic delivery would lead to a globally impaired immune system. Delivery systems and reagents that selectively target and block cell adhesion to prevent pathological inflammation have been sought.

The repertoire of leukocyte types and receptor-ligand interaction described for inflammatory responses are also involved in autoimmune diseases [rheumatoid arthritis (RA), multiple sclerosis (MS), Graves disease, Crohn's disease (CD), AIDS, diabetes, graft-versus-host disease (GVHD), inflammatory bowel disease (IBD)] and rejection of allograft tissues/organs.

Autoimmune and allograft rejection responses are distinguished by the recruitment of T-cells and the development of a specific/adaptive immune response. Integrin interactions with ligands play a key role in recruiting circulating T-cells to extravascular sites where autoimmune and allograft rejection occurs. In the case of T-cells, extravascular infiltration is critical for antigen recognition, clonal expansion of specific antigen-responsive T-cells, and the destructive attack of cytotoxic T-cells on antigen-bearing tissues. These specific receptor-ligand interactions represent therapeutic targets for suppressing pathologic adaptive immune responses, and therapeutic strategies have been sought to modify receptor-ligand interactions in therapy of autoimmune diseases and allograft rejection.

New reagents and methods for treating and preventing excessive inflammation, autoimmune diseases, tissue rejection, cancer metastasis and other pathological conditions preceded by the binding of an integrin receptor with its ligand are being sought.

### BRIEF DESCRIPTION OF THE FIGURES

FIG. 1 schematically represents the anti-inflammatory/immunosuppressant action of the bio conjugates of the present invention. The normal immune response to vascular injury and the response of the injured site in the presence of the biospecific bioconjugates are illustrated. The diagram shows the biointerface formed by the bioconjugates of the present invention creating a physical barrier against subsequent inflammatory cell adhesion.

FIG. 2 is a reaction scheme for the preparation of a preferred embodiment of the present invention, a dextran-peptide bioconjugate.

FIG. 3 is a nuclear magnetic resonance representation of dextran.

FIG. 4 illustrates the results of an adhesion assay of a bioconjugate of the present invention with bovine endothelial cells stimulated to express the integrin ligand ICAM-1. In this assay, the bioconjugate effectively bound to endothelial cells, reducing monocyte adhesion to levels observed in control, non-stimulated cells.

### SUMMARY

Bioconjugates capable of preventing cellular interactions mediated by integrin/ligand binding have been discovered. When administered to an individual, the bioconjugates form a cell adhesion barrier in a target tissue that prevents and treats the pathological conditions preceded by cellular interactions. The bioconjugates comprise a hydrophilic polymer and a peptide wherein the peptide preferably comprises at least the binding site of an integrin for a ligand expressed on a cell. When applied to a living tissue, the bioconjugates bind specifically to cells expressing the ligand and form a blockade or biofilm that prevents subsequent cell binding at the blocked tissue. Pathological consequences of cellular interactions, which include inflammation, autoimmune diseases, tissue rejection, cancer metastasis and other pathological conditions preceded by cellular interactions, are thus prevented.

The therapeutic bioconjugate includes a hydrophilic polymer; and one or more peptides capable of binding specifically to a ligand expressed on a cell surface. The bioconjugate blocks interactions between cells in a living tissue when the ligand is expressed on the surface of at least one of said cells. Moreover, the bioconjugate can block interaction between a cell and an extracellular matrix wherein said ligand is capable of binding to a component of said matrix. The bioconjugate is intended to block pathological reactions triggered by cellular interactions in a living tissue.

In some embodiments, the bioconjugate has a peptide that includes the amino acid sequence of the binding portion of an integrin for a tissue-bound ligand. The bioconjugate may have blocking cell signaling receptors implicated in the regulation of cellular adhesion, migration, tumor metastasis, proliferation, angiogenesis, bone resorption, apoptosis, or gene expression. Among these are the binding portion of an integrin a subunit or an integrin β subunit. These binding portions of the integrin subunits include SEQ ID NOS 1-202. The bioconjugate's binding portion can be, for example, a portion of the integrin α₂ subunit (CD49b, VLA-2, platelet gpla) I domain, integrin α₄ (CD49b, VLA-4), integrin α₅ (CD49e, VLA-5), integrin α_{L} (CD11a) I domain, integrin α_{M} subunit (CD11b) I domain, integrin α_{I1b} I domain, integrin α_{I1b} (CD41) heavy chain, integrin α_{11b} (CD41) light chain, integrin β₁ (CD29) subunit, the integrin β₂ (CD18) subunit, integrin β₃ (CD61) subunit, or integrin β₇ (LPAM-1) subunit.

In one embodiment, the bioconjugate's peptide includes the portion of the integrin α₂ subunit (CD49b, VLA-2, platelet gpla) I domain that binds specifically to ligands CN I, CN II, CN III, CN IV, LN and/or the echovirus-1 receptor. In another embodiment, the bioconjugate's peptide is a portion of the integrin α₄ (CD49b, VLA-4) subunit that binds specifically to the ligands VCAM-1, FN, MAdCAM-1, TSP and/or invasin. In yet another embodiment, the bioconjugate's peptide is a portion of the integrin α₅ (CD49e, VLA-5) that binds specifically to ligands FN, L1 or invasin. In other embodiments, the bioconjugate's peptide is a portion of the integrin α₁ (CD11a) I domain that binds specifically to the ligands ICAM-1, ICAM-2, ICAM-3 or LPS. In other embodiments, the bioconjugate's peptide is a portion of the integrin α_{M} subunit (CD11b) I domain that binds specifically to the ligands iC3b, ICAM-1, ICAM-2, ICAM-4, Fb, Factor X, CD23, NIF, heparin, beta glucan, or LPS. In other embodiments, the bioconjugate's peptide is a portion of the integrin α_{I1b} (CD41) heavy chain that binds specifically to the ligands Fb, FN, VN, TSP or vWF. In other embodiments, the bioconjugate's peptide is a portion of the integrin α_{11b} (CD41) light chain that binds specifically to the ligands Fb, FN, VN, TSP and vWF. In another embodiment, the bioconjugate's peptide is a portion of the integrin β₁ (CD29) subunit that binds specifically to the ligands FN, LN, CN, VCAM-1, FN, MAdCAM-1, TSP or invasin. Moreover, the bioconjugate's peptide can be a portion of the integrin β₂ (CD18) subunit that binds specifically to the ligands ICAM-1, ICAM-2, ICAM-3, ICAM-4, LPS, iC3b, Fb, Factor X, CD23, NIF, heparin, and/or betaglucan. In another embodiment, the bioconjugate's peptide is a portion of the integrin β₃ (CD61) subunit that binds specifically to ligands fibrinogen, fibronectin, vitronectin, thrombospondin, von Willebrand factor, osteopontin, bone sialoprotein, laminins, collagens, and/or neural cell adhesion molecule L1.

In another embodiment, the bioconjugate's peptide is a portion of the integrin β₇ (LPAM-1) subunit that binds specifically to the ligands VCAM-1, fibronectin, MAdCAM-1, or E-cadherin (cadherin-1).

This invention also includes the nucleic acids coding for peptides of the peptide portion of the bioconjugates. The nucleic acid sequences are provided in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 86, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 186, 185, 187, 189, 191, 193, 195, 1197, 199 and 201.

This invention also includes the peptides for preparation of bioconjugate having their sequence set out in P-2, P-49 and SEQ ID NOS 1-218 and modified with an additional N-terminal or C-terminal cysteine residue. In another embodiment, the above nucleic acid sequences are modified to accommodate the additional cysteine residue(s).

The bioconjugates also include a polymer, that can be a polysaccharide or an oligosaccharide. In another embodiment, the polymer is derived from a polysaccharide or an oligosaccharide by the addition of chemical groups capable of reacting with a peptide to form said bioconjugate.

In another embodiment, the bioconjugate has the formula XY_{b} wherein X is a low cell-adhesive, hydrophilic polymer, Y is a peptide comprising a portion of the binding site of an integrin for a ligand expressed on a cell surface, and b is greater than 0. In another embodiment, the polymer X is a polysaccharide or an oligosaccharide. In another embodiment X is a derivative of a polysaccharide or of an oligosaccharide in which the derivative saccharide has reactive groups such that the derivative saccharide reacts with peptides to form the bioconjugate. The reactive group can be a hydroxyl group. In other embodiments, the polysaccharide or oligosaccharide can be agarose, dextran, heparin, chondroitin sulfate, hydroxyethyl starch, and hyaluronic acid. More preferably, the polymer is a dextran and thed peptide is the binding portion of an integrin. In other embodiments, the polymer is polyvalent and is, for example, poly(ethylene glycol), poly(ethylene oxide), poly(vinyl alcohol), poly(acrylic acid), poly(ethylene-co-vinyl alcohol), poly(vinyl pyrrolidone), poly(ethyloxazoline), and/or poly(ethylene oxide)-co-poly(propylene oxide) block copolymers. In other embodiments, the polymer can be copolymers, block copolymers, graft copolymers, alternating copolymers, or random copolymers. Preferably, the polymer is essentially inert. Preferably, the polymer is degradable by hydrolytic or enzymatic means. Examples of degradable polymer are one or more blocks consisting of lactic acid, glycolic acid, ε-caprolactone, lactic-co-glycolic acid oligomers, trimethylene carbonate, anhydrides, and amino acids. In one embodiment, the polymer is a serum protein, such as albumin

In other embodiments, the bioconjugate is in a pharmaceutically acceptable carrier. Alternatively, the bioconjugate is immobilized on a solid substrate. Preferably, the bioconjugate is immobilized on an implantable medical device. The bioconjugate could be immobilized on a drug delivery device or an in vitro diagnostic device.

In other embodiments, there is provided a kit including one or more bioconjugates as well as reagents and apparatus suitable for administering the bioconjugate to an individual. Alternatively, the bioconjugate can be in a pharmaceutically acceptable carrier.

In one embodiment, there is formed on a mammalian tissue a biointerface such that the biointerface includes a plurality of bioconjugates bound to a plurality of ligands on the tissue.

There also is provided a method of preparing a bioconjugate including the steps of providing a hydrophilic polymer having one or more reactive groups, providing a bioselective peptide comprising a chemical group capable of reacting with the reactive groups, and contacting the polymer and the peptide under conditions such that the reactive and chemical groups react to form the bioconjugate. In another embodiment, the reactive groups of the polymer are hydroxyl groups and the chemical group of the peptide is a sulfhydryl group. In preferred embodiments, the polymer is a polysaccharide, such as activated dextran or hydroxyl starch.

In other embodiments the peptide of the bioconjugate is selected from the group consisting of SEQ ID NOS 7-14, 25-32, 35-38, 43-48, 55-56, 65, 66, 93, 94, 97, 98, 107-110, 119-124, 133-136, 141, 142, 153, 154, 157-164, 171-174, 179-200, 203-212, 215 and 216, the peptide comprising a cysteine residue. In other embodiments, the peptide is selected from the group consisting of SEQ ID NOS 1-218, the peptide including additionally an N-terminal or a C-terminal cysteine residue.

In other embodiments, there is provided a method of preparing a bioconjugate including the steps of providing a peptide selected from the group consisting of SEQ ID NOS 1-218, modifying the peptide by addition of an N-terminal or C-terminal cysteine residue, providing an amount of activated dextran, and contacting the activated dextran and the modified peptide under conditions, whereby the dextran and the modified peptide react to form the bioconjugate.

There is also provided a method for preventing adhesion of a mobile cell to a cell immobilized on a substrate including the step of applying a bioconjugate specific for the immobilized cell under such conditions that the bioconjugate forms a cell adhesion barrier on the immobilized cell and prevents adhesion of the mobile cell.

There also is provided a method of blocking pathological reactions triggered by cellular interactions in a living tissue. This method has the step of administering to the living tissue a bioconjugate selective for a target tissue, whereby the bioconjugate forms a cell adhesion barrier at a targeted tissue site. In other embodiments, the bioconjugate is the binding portion of an integrin for its ligand expressed on the target tissue. In other embodiments, the bioconjugate is administered intravascularly, orally, intramuscularly, intraperitoneally, subcutaneously, cerebrospinally, endovascularly, rectally or topically. When the bioconjugate is administered intravascularly in a biologically compatible solution, it is administered at a concentration of between about 1 μg/L and 100 g/L. Preferably the bioconjugate is administered to an individual in a pharmaceutically acceptable composition. Preferably, the amount of administered bioconjugate is between about 1-1000 mg/kg body weight.

In another method of preventing and treating thrombosis, an anti-coagulating amount of a bioconjugate having one or more peptides capable of binding selectively to integrin ligands expressed on inflamed endovascular cells is administered to tissue containing the inflamed endovascular cells. In other embodiments, the integrin ligands are CN I-IV, LN, or the Echovirus-1 receptor. In other embodiments, the bioconjugate's peptide is selected from the group consisting of P-2, P-49, and SEQ ID NOS 1, 2, 3-8, 91-106, 129-192, 203 and 204.

Also provided is a method of preventing and treating atherosclerosis. An anti-atherosclerotic-effective amount of the bioconjugate including one or more peptides capable of binding selectively to integrin ligands expressed on or around atherosclerotic cells is administered to tissue containing the atherosclerotic cells. In other embodiments, the integrin ligands are VCAM-1, FN, MAdCAM-1, TSP, invasin or a combination thereof. In other embodiments, the bioconjugate's peptide is selected from the group consisting of P-49 and SEQ ID NOS 9-38, 59-106, 129-202 and 207-210.

Also provided is a method of claim 57 for preventing and treating systemic inflammatory response syndrome. An effective amount of the bioconjugate comprising one or more peptides capable of binding selectively to integrin ligands expressed on cells in such inflamed tissue is administered to the tissue. In other embodiments, the integrin ligands are FN, L1 or invasin. The bioconjugate's peptide(s) is selected from the group consisting of P-49 and SEQ ID NOS 9-38, 59-106, 129-202 and 207-210.

In the method of preventing and treating multiple organ failure (MOF), a MOF-effective amount of the bioconjugate having one or more peptides capable of binding selectively to integrin ligands expressed on cells in affected tissue is administered to the tissue. In other embodiments, the integrin ligands are ICAM-1, ICAM-2, ICAM-3, LPS or a combination thereof. The bioconjugate's peptide(s) is selected from the group consisting of P-49 and SEQ ID NOS 39-58, 107-128 and 211-218.

In the method of preventing and treating autoimmune disease, an effective amount of a bioconjugate including one or more peptides capable of binding selectively to integrin ligands expressed on cells implicated in the autoimmune disease is administered to tissue containing the cells. In other embodiments, the integrin ligand is VCAM-1, FN, MAdCAM-1, TSP, invasin, ICAM-1, ICAM-2, ICAM-3, LPS, iC3b, ICAM-1, ICAM-2, ICAM-4, Fb, Factor X, CD23, NIF, heparin, β-glucan, LPS, FN, Fb, CN I, VN, FN, LN, CN, Fb, Factor X, CD23, NIF, heparin, β-glucan or a combination thereof. The bioconjugate's peptide(s) are selected from the group consisting of P-2, P-49 and SEQ ID NOS 1-218.

In the method of preventing and treating inflammatory diseases, an effective amount of a bioconjugate comprising one or more peptides capable of binding selectively to integrin ligands expressed on cells of inflamed tissue is administered to a tissue containing the inflamed cells. The integrin ligand may be CN I-IV, LN, Echovirus-1 receptor, VCAM-1, FN, MAdCAM-1, TSP, Invasin, L1, LPS, ICAM-1-4, iC3b, Fb, Factor X, CD23, NIF, heparin, β-glucan, VN, vWF or a combination thereof. The bioconjugate's peptide(s) is selected from the group consisting of P-2, P-49, and SEQ ID NOS 1-202 and 205-219.

In a method of preventing and treating allograft transplant rejection, an anti-rejection amount of a bioconjugate having one or more peptides capable of binding selectively to integrin ligands expressed on T cells implicated in allograft transplant rejection is administered to an individual having transplanted tissue. The integrin ligand may be VCAM-1, FN, MAdCAM-1, TSP, invasin, ICAM-1-4, LPS, iC3b, Fb, Factor X, CD23, NIF, heparin, β-glucan, LN, CN, vWF, OP, BSP, L1 and E-cadherin. The bioconjugate's peptide(s) may be any of P-49 and SEQ ID NOS 9-30, 39-58, 91-200 and 211-218. Transplant rejection also may be concurrently treated with an Immunosuppressant, such as cyclosporine.

In a method of preventing and treating Crohn's disease, an effective amount of the bioconjugate comprising one or more peptides capable of binding selectively to integrin ligands expressed on inflamed cells in gut tissue is administered. The integrin ligand may be VCAM-1, FN, MAdCAM-1, TSP, invasin, ICAM-1-4, iC3b, Fb, Factor X, CD23, NIF, heparin, β-glucan, CN I, VN, LN, OP, BSP, L1, vWF and/or E-cadherin. The bioconjugate may have one or more peptides selected from the group consisting of P-49 and SEQ ID NOS 9-30, 30-58, 93-200 and 211-218.

In a method of preventing and treating inflammatory bowel disease, an effective amount of a bioconjugate includes one or more peptides capable of binding selectively to integrin ligands expressed on inflamed cells in gut tissue is administered. The bioconjugate has one or more peptides selected from the group consisting of P-49 and SEQ ID NOS 9-30, 39-58, 91-200 and 21-218.

In a method of preventing and treating sequelae of a bacterial infection, an effective amount of the bioconjugate comprising one or more peptides capable of binding selectively to integrin ligands expressed on secretory membranes is administered. The bioconjugate has one or more peptides selected from the group consisting of P-49 and SEQ ID NOS 39-58, 107-192 and 211-216.

In a method of preventing and treating sepsis or septic shock, an effective amount of a bioconjugate comprising one or more peptides capable of binding selectively to integrin ligands such as LFA-1, ICAM-1, VCAM-1 and a combination thereof is administered. The bioconjugate includes one or more peptides selected from the group consisting of P2, P-49 and SEQ ID NOS 1-30, 39-58, 91-200 and 211-18.

In a method of preventing and treating ischemia-reperfusion injury, an effective amount of a bioconjugate comprising one or more peptides capable of binding selectively to integrin ligands is administered intravenously. The bioconjugate includes one or more peptides selected from the group consisting of P-49 and SEQ ID NOS 9-30 and 39-218.

In a method of preventing and treating cancer metastasis, an anti-metastasis effective amount of the bioconjugate comprising one or more peptides capable of binding selectively to integrin ligands is administered systemically to an individual or locally to tissue containing or suspected of containing cancer. The bioconjugate includes one or more peptides selected from the group consisting of P-49 and SEQ ID NOS 91, 92, 203 and 204.

In a method of treating conditions caused by viper and rattlesnake bites, an anti-venom-effective amount of the bioconjugate having one or more peptides capable of binding selectively to at least one integrin ligand on a bitten tissue site is administered. In some embodiments, the bioconjugate has a peptide of SEQ ID NOS 153 and 154.

Also embodied herein are therapeutic replacement fluids including a bioconjugate and a pharmaceutically acceptable diluent.

### DETAILS OF THE INVENTION

We have created a family of bioselective bioconjugates that specifically bind to ligands expressed during cell-cell interactions including immune responses that result in pathology. The bioconjugates selectively target and bind to tissue surfaces, forming a protective barrier against pathologically driven cell-cell interactions. The bioconjugates, provided systemically or locally, selectively target tissues to suppress pathologically excessive damage to healthy tissues and thus limit deleterious outcomes. The various bioconjugates may be used in the prevention and therapy of a number of pathological processes involving leukocyte adhesion to tissue surfaces, including but not limited to, inflammation, septic shock, post-trauma multiple organ failure, ischemic reperfusion injury, transplant rejection, infectious inflammatory diseases, and autoimmune diseases. Other pathological responses that are the result of cell-cell interactions that may be therapeutically treated by the present bioconjugates include, but are not limited to, thrombosis, atherosclerosis, cancer metastasis, autoimmune diseases, hookworm infection, bacterial and viral infection, and the sequelae of viper and rattlesnake bites.

The term “bioconjugate” as used herein means a compound in which at least two components, a peptide and a cell-adhesion-barrier polymer are chemically attached, i.e., conjugated. Methods of conjugation of the bioselective peptide and the cell adhesion barrier molecules are generally known in the art. The specific conjugation method is determined by the choice of cell adhesion barrier molecule and the accepted linking methods to the selected bioselective molecule, preferably a protein or peptide. Both univalent and multivalent conjugation methods are suitable. The conjugation method is selected to produce a bioconjugate that retains the bioselective and blockade abilities of the bioconjugate. In preferred embodiments of the invention, the molecules are attached in vitro prior to application to the living tissue. In certain other embodiments the molecules may be designed with appropriate linking groups that cause them to congregate in vivo.

As used herein “bioselective” means a molecule that (a) is capable of binding specifically to its ligand, preferably an integrin ligand; (b) is physiologically compatible with living tissue; (c) is generally chemically inert; and (d) exhibits little or no binding affinity for cellular components other than the targeted ligand. Peptides having the amino acid sequence based on the ligand binding site of the integrins have a selective affinity for the targeted ligand, e.g., provide the targeting ability of the bioconjugates for tissue such as injured or diseased tissue that express the ligand. Since normal tissue does not generally express these ligands (or expresses ligand in low quantity), the bioselective bioconjugates may be delivered systemically as well as locally as therapeutic agents to suppress inflammation where these ligands are expressed and to prevent the pathological consequences of excessive tissue inflammation.

As used herein, the term “integrin ligand” means the moiety on a specific cell type that binds to surface-bound integrins during the course of cellular interactions. Integrin ligands are the target binding site for the bioconjugates of the present invention. Each bioconjugate comprises one or more peptides that bind specifically to one or more particular cell-surface expressed ligands and also comprises a low-adhesive polymer. The bound bioconjugates block binding at the ligand to any subsequent cell surface integrin by forming a blockade or an “internal tissue bandage” that prevents specific, unwanted cell-cell interactions.

The term “peptide” is used herein in its broadest sense to refer to a sequence of subunit amino acids, amino acid analogs, or peptidomimetics. Peptides may be linked, for example, by peptide bonds, to form polypeptides.

The term “biointerface” as used herein means a collection of bioconjugates of the present invention bound to their ligand on a cell surface. When a bioconjugate binds to its ligand, an essentially inert blockade results, and subsequent interaction between cells is prevented.

The term “cell adhesion” as used herein means the binding of at least one cell to another cell or to a component of an extracellular matrix.

The term “cell adhesion barrier” as used herein means the biointerface that forms in situ in a tissue as a result of bioconjugate binding. Cell adhesion barrier molecules have properties that intrinsically inhibit cell adhesion by forming a physical barrier to cell-cell/tissue adhesion when applied to cell, tissue, or biomaterial surfaces. The cell adhesion barrier prevents adhesion of circulating cells to a cell surface, a component of an extracellular matrix or another material.

The term “polyvalent polymer” as used herein means a polymer having more than one reactive group at which a peptide or other moiety may be chemically linked to the polymer. In preferred embodiments of this invention, the reactive groups are hydroxyl groups that react with the sulfydryl groups on a peptide to form the bioconjugate. The polyvalency of the polymer provides the opportunity to make a bioconjugate comprising multiple connections of a peptide to the polymer or multiple peptides, which may be the same or different.

The therapeutic bioconjugates of the present invention comprise a polymer that forms the cell adhesion barrier. Preferably the polymer is multivalent, i.e., contains multiple reactive groups to allow a high number of peptides to be incorporated into the bioconjugate. In certain preferred embodiments, the polymer component is a hydrophilic polymer that is highly soluble in aqueous solutions.

The therapeutic bioconjugates of the present invention also comprise one or more peptides that selectively and strongly bind cell ligands and effectively immobilize the polymeric component at a tissue surface. Tissue ligands are typically in high enough concentrations on tissue surfaces to promote high-density surface binding of bioconjugates, creating a polymer barrier to cell adhesion on ligand-presenting surfaces. The polymeric barrier is a biointerface on a tissue surface that blocks subsequent binding of circulating cells to the tissue surface.

The therapeutic bioconjugates of the present invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

The bioconjugates are preferably prepared by contacting a cell-adhesion-barrier polymer having multiple reactive chemical groups with a peptide having multiple chemical reactive groups under conditions where the polymer and peptide react to form covalent bonds.

Disclosed herein is a method for synthesis of a preferred embodiment of the present invention, bioconjugates comprising dextran and one or more peptides having the amino acid sequence of a portion of the integrin binding site. In a preferred method, dextran containing multiple hydroxyl groups is reacted directly with peptide functional groups (usually SH or S—S) to form covalently bound peptide in the dextran bioconjugate. Generally, the reaction is conducted at a temperature and a time such that (1) the solvent is in liquid form, (2) the dextran and the peptide do not degrade, and (3) detectable levels of product is obtained. Preferably, this reaction is conducted in the presence of a suitable solvent, e.g., water, under atmospheric conditions and pH optimal for formation. Upon completion of the reaction, the resulting bioconjugate of activated dextran and covalently attached peptide is recovered by conventional methods including, but not limited to, neutralization, extraction, precipitation, chromatography, filtration and the like.

Another preferred method for preparing the bioconjugates is presented. In this method a polymer having multiple reactive chemical groups is contacted with linker molecules containing two or more chemical reactive groups under conditions whereby the two compounds react to form covalent bonds. The polymer with covalently bound linker molecules is then contacted with a peptide with multiple chemical reactive groups under conditions whereby the two components react to form covalent bonds and the final therapeutic bioconjugate product.

Also disclosed is a method for synthesis of a preferred embodiment of the present invention, bioconjugates comprising dextran and one or more peptides having the amino acid sequence of the binding site of an integrin. In this method, dextran is first activated by reaction with a linking molecule, preferably dimethylaminopyridinine (DMAP). Generally, this reaction is conducted at a temperature and time range such that (1) the solvent is in liquid form, (2) the cell adhesion barrier polymer, (3) the linking molecule do not degrade, and (4) detectable levels of product are obtained. Preferably, the reaction is conducted in the presence of a suitable solvent, e.g., DMSO, under atmospheric conditions optimal for product formation. Upon completion of the reaction, the resulting conjugate containing the cell adhesion barrier polymer with covalently attached linking molecules, e.g., activated dextran, is recovered by conventional methods such as neutralization, extraction, precipitation, chromatography, filtration and the like. The multiple functional groups of activated dextran react with a sulfhydryl group, preferably on a cysteine residue in the peptide. Upon completion of the reaction, the resulting bioconjugate containing dextran with covalently attached peptide is recovered by conventional methods including, but not limited to, neutralization, extraction, precipitation, chromatography, filtration and the like.

Peptides are presented that may be used in the synthesis of the present bioconjugates. The peptides preferably comprise the amino acid sequence of the binding site of an integrin specific for a targeted ligand expressed on a cell surface. The peptides also comprise one or more sulfhydryl groups provided, generally, by cysteine residues. Certain of the peptides comprising amino acid sequences of binding sites of the integrins naturally comprise cysteine. Other preferred peptides may be modified for use in the synthetic methods by the addition of N-terminal or C-terminal cysteine residues. Preferred peptides for use in the preparative methods of the present method are members of the group consisting of SEQ ID NOS 1-112, with a cysteine residue added to the N- or C-terminus of peptide sequences which do not naturally have cysteine. The peptides described herein may be isolated from a naturally occurring protein, may be chemically synthesized, or may be recombinantly expressed by methods well known in the art. Nucleic acids for recombinant preparation of the peptides are presented in SEQ ID NOS 113-225.

Table 1 (at end) presents the amino acid sequence of the peptides, the nucleic acid sequence corresponding to each peptide, the integrin from which the peptide is derived, the target ligand for each peptide and therapeutic administration of the preferred bioconjugates of the present invention.

From Table 1 it can be seen that the bioconjugates of the present invention may be used therapeutically in a large number of diseases and disease states caused by pathological consequences of cell-cell interactions through integrin/ligand binding. Many of these diseases involve inflammation at various tissue sites as, for example, Crohn's disease, intestinal bowel disease, multiple organ failure (MOF), systemic inflammatory response, and septic shock. Other diseases that are the pathological consequences of intercellular reactions mediated by integrins and may be therapeutically treated by the bioconjugates of the present invention include, but are not limited to allograft transplant rejection, cancer metastasis, bacterial or viral infection, thrombosis, atherosclerosis, ischemia-reperfusion injury, autoimmune diseases, and hookworm infection.

The above table is a compendium of known integrin/ligand pairs and illustrates the therapeutic applications of bioconjugates comprising these known integrins. However, it is anticipated that as new integrins are discovered and characterized, they may likewise be used as sources of peptides in the bioconjugates of the present invention and will find therapeutic use in preventing and treating disease states in which integrin/ligand binding is implicated.

In certain embodiments of the present invention, peptides other than those derived from integrins may be used to form cell adhesion barriers. Thus, for example, bioconjugates synthesized from a barrier polymer and antibodies or antibody fragments capable of binding to selected antigens expressed on a cell surface, an extracellular matrix or tissue surface may likewise be used in the methods of the present invention to prevent or treat diseases triggered by cellular interactions.

The therapeutic bioconjugates of the present invention bind to a specific target tissue. This specificity is achieved by selecting the peptide component of the bioconjugate that specifically binds to ligands that are expressed on cells in selected tissues, not generally on cells circulating in the bloodstream. A bioconjugate capable of binding to circulating cells might create aggregates in the bloodstream which could compromise blood flow. Examples of ligands expressed on non-circulating-cell surfaces include, but are not limited to, CN I, CN II, CN III, CN IV, LN, Echovirus-1 receptor, VCA, FN, L1, invasin, MAdCAM-1, TSP, ICAM-1, ICAM-2, ICAM-3, ICAM-4, iC3b, Fb, Factor X, CD23, NIF, heparin, β-glucan, LPS, VN, vWF, FN, LN, CN, VCAM-1 and MAdCAM-1. The definition of these abbreviations are given at the end of Table 1.

In an important aspect of the present invention, pharmaceutical compositions comprising one or more bioconjugates of the present invention and a pharmaceutically acceptable carrier are presented. The pharmaceutical combinations and methods of this invention are adapted to therapeutic use as agents in the treatment or prevention of pathological excessive leukocyte adhesion/infiltration and subsequent tissue injury according to the methods described herein. The bioconjugates may be suspended in aqueous solution, e.g., saline solution, for intravenous delivery of the therapeutic compounds.

The compounds of the present invention are generally administered in the form of a pharmaceutical composition comprising at least one of the bioconjugates of this invention together with a pharmaceutically acceptable carrier or diluent. Thus, the compounds of this invention can be administered either individually or together in any conventional oral, or parenteral dosage form.

For oral administration the pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Fillers in soft and hard-filled gelatin capsules have preferred materials, including lactose or milk sugar as well as high molecular weight polyethylene glycols. When aqueous suspensions and/or elixirs are desired for oral administration, the bioconjugates of this invention can be combined with various sweetening agents, flavoring agents, coloring agents, emulsifying agents and/or suspending agents, as well as such diluents as water, ethanol, propylene glycol, glycerin and combinations thereof.

The bioconjugates of this invention may also be administered in a controlled release formulation such as a slow release or a fast release formulation. Such controlled release dosage formulations of the combination of this invention may be prepared using methods well known to those skilled in the art. The method of preferred administration will be determined by the attendant physician or other person skilled in the art after an evaluation of the subject's condition and requirements.

For purposes of parenteral administration, solutions in sesame or peanut oil or in aqueous propylene glycol can be employed, as well as sterile aqueous solutions of the water-soluble salts and sugars. Such aqueous solutions may be suitably buffered, if necessary, and the liquid diluent first rendered isotonic with sufficient saline or dextrose. These aqueous solutions are especially suitable for intravenous, intramuscular, subcutaneous and intraperitoneal injection. In this connection, the sterile aqueous solutions are all readily obtainable by standard techniques well known to those skilled in the art.

Methods of preparing various pharmaceutical compositions with a certain amount of active ingredient are known, or will be apparent in light of this disclosure, to those skilled in this art.

The present invention also relates to pharmaceutical compositions in kit form. The kit may include one or more pharmaceutical compositions. The kit includes container means for containing the compositions. Typically the kit includes directions for the administration of the compositions. The kit form is particularly advantageous when the separate components are administered in different dosage forms (e.g., oral and parenteral) or are administered at different dosage concentrations as desired by the prescribing physician.

In an important aspect of the present invention, improved biomedical devices are presented. The devices are improved by the incorporation of one or more bioconjugates of the present invention disposed on or in the biomedical device.

As used herein, a “biomedical device” refers to a device to be implanted into or attached to a tissue in a subject, for example, a human being, in order to bring about a desired result. Particularly preferred improved biomedical devices according to this aspect of the invention include, but are not limited to catheters coated with the present bioconjugates to prevent localized inflammation around the biodevice. Similarly, wound dressings are biomedical devices that may be improved by coating with the present bioconjugates and then applied to inflamed surfaces.

As used herein, “disposed on or in” means that the one or more bioselective bioconjugates can be either directly or indirectly in contact with an outer surface, an inner surface, or embedded within the biomedical device. “Direct” contact refers to disposition of the bioconjugates directly on or in the device, including, but not limited to, soaking a biomedical device in a solution containing the one or more bioconjugates, spin coating or spraying a solution containing the one or more bioconjugates onto the device, implanting a device that would deliver the bioconjugate, and administering the bioconjugate through a catheter directly on to the surface or into any organ or transplant.

“Indirect” contact means that the one or more bioconjugates do not directly contact the biomedical device. For example, the one or more bioconjugates may be disposed in a matrix, such as a gel matrix or a viscous fluid, which in turn is disposed on the biomedical device. Such matrices can be prepared to, for example, modify the binding and release properties of the one or more bioconjugates as required.

Exact dosing of bioconjugate therapy depends on many factors, among them the binding affinity of a particular bioconjugate for the targeted tissue ligands and the rate at which the bioconjugate is cleared from targeted tissue sites. Binding affinity of the bioconjugate for tissue ligands affects the amount of local tissue requirements for maintaining saturated coverage of bioconjugate on ligand-expressing tissue. Two major factors affect binding affinity: 1) the number of ligand-binding peptides per conjugate molecule; and 2) the affinity of the complexed peptide for the targeted ligand. The rate at which the bioconjugate is cleared from targeted tissue sites is dependent in part on the turnover rate of cells presenting tissue ligands. The turnover rate is driven by a constant internalization of surface molecules, and ligand internalization rate determines the duration of the ligand-bound bioconjugates on cell/tissue surfaces. The amount of bioconjugate delivered to a particular tissue in an individual in need of therapy varies by size of person, affinity of the peptide of the bioconjugate for the target ligand, turn-over rate of cells at the specific stage of disease at the time of administration and the mode of administration. It is anticipated that continuous or multiple administrations of bioconjugate will be most effective in treating and controlling the progress of disease.

In an important aspect of the present invention, methods are given for treating diseases caused by the pathological reactions triggered by interaction between different cell types in a living tissue. The methods comprise the step of administering to a subject in need thereof an amount of a bioselective bioconjugate of the present invention effective to block target ligands and thereby suppress subsequent cell-cell interaction and prevent the diseases.

In the methods of the present invention, the therapeutic bioselective bioconjugates may be administered by targeted delivery or by localized delivery. As used herein “targeted delivery” means systemic delivery of the present bioconjugates to an internal inflamed tissue surface. The biospecific bioconjugates target tissue surfaces with selected ligands and thus are agents of targeted delivery.

As used herein “localized delivery” means, for example, the direct application of the present bioconjugates to an exposed tissue surface. Topical application to a wound or inflamed burned tissue, for example, would be most suitable for localized delivery. Delivery systems such as aerosols or swabs may be used in localized delivery to other tissue or mucosal surfaces. Intra-arterial delivery of bioconjugate to a particular organ also is contemplated.

Therapy of Inflammation in Tissue

It has been discovered that the normal response to vascular injury may be suppressed by certain therapeutic bioconjugates that selectively target and locally bind to inflamed tissue surfaces that express certain ligands, such as ICAM-1. The bound bioconjugates form a protective barrier against abnormally excessive leukocyte adhesion/infiltration and subsequent tissue injury. The effective blockade suppresses the pathological consequences of excessive leukocyte adhesion/infiltration into vulnerable tissue.

To exemplify the biospecific activity and adhesion of the bioconjugates of the present invention, the characteristics of a preferred embodiment, the dextran/ICAM-1-binding A domain peptide conjugates, to inflammatory cells were measured as described in Experiments 2 and 3 hereinbelow.

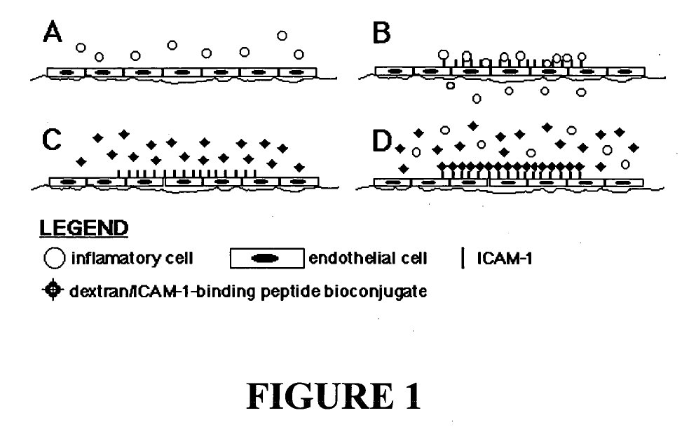

FIG. 1 depicts the reaction of bioselective dextran bioconjugate at inflamed endothelial cells expressing ICAM-1. In FIG. 1, the intravascular action of the present bioconjugates is illustrated. In FIG. 1, the lumen of the vessel and circulating blood/fluid volume are illustrated above the endothelial layer; the vessel wall is below the endothelium. FIG. 1 (A) illustrates a normal blood vessel in uninjured tissues with circulating polymorphic neutrophils (PMNs). FIG. 1 (B) illustrates inflamed (ICAM-1-expressing) endothelial cells following tissue injury. PMNs bind to ICAM-1 on inflamed endothelial cells and invade the vessel wall and surrounding tissues. Traumatic shock can induce excessive PMN adhesion and activation resulting in damage to healthy tissues and multiple organ failure (MOF). FIG. 1 (C) illustrates an inflamed blood vessel immediately after infusion of resuscitative fluids containing dextran/ICAM-1-binding peptide bioconjugate of the present invention. FIG. 1 (D) illustrates binding of dextran bioconjugate to inflamed endothelial cells forming a non-adhesive barrier to PMNs. Invasion of PMNs into healthy tissues is thus reduced. Other leukocytes that interact with ICAM-1 are also blocked by this therapeutic strategy. Other endothelial cell surface ligands, e.g., VCAM-1, could also be targeted using peptides that selectively bind to other endothelial cell surface ligands.

Methods are presented for suppressing inflammation in a tissue. In certain instances, an inflamed tissue is contacted locally with one or more bioconjugates in an amount effective to inhibit tissue/leukocyte binding and suppress inflammation. The topical methods may also be used to enhance healing of inflamed flesh wounds caused by trauma or heat. In other instances the bioselective bioconjugates are delivered systemically to target the inflamed tissue sites. These methods are useful for preventing and treating inflammatory diseases including chronic inflammation of gut, cervix, eyes and lung.

In preferred methods for preventing and treating inflammatory diseases, an anti-inflammation-effective amount of a bioconjugate comprising one or more peptides capable of binding selectively to integrin ligands expressed on tissues containing the inflamed cells is applied to inflamed tissue such as such as gut, cervix, eyes, lung and inflamed flesh wounds. In these methods the bioconjugate comprises peptides capable of binding to the target ligands expressed on inflamed tissue cells. Most preferably the bioconjugate comprises one or more peptides selected from the group consisting of P6-P16, P21-P30, P48-P104, P109-P112 (Table 1).

In preferred methods for preventing and treating systemic inflammatory response syndrome (SIRS), there is administered an anti-SIRS-effective amount of bioconjugate comprising one or more peptides capable of binding selectively to integrin ligands expressed on cells in inflamed tissue. Preferably, the bioconjugate comprises peptides capable of binding to a target ligand from the group shown in Table 1. Most preferably the bioconjugate comprises one or more peptides selected from the group consisting of P1-99, P104 and P106-112 (Table 1).

In preferred methods for preventing and treating inflammatory bowel disease (IBD), an anti-IBD-effective amount of bioconjugate comprising one or more peptides capable of binding selectively to target ligands expressed on cells in inflamed bowel tissue is applied to the tissue. Preferably, the bioconjugate comprises peptides capable of binding to an integrin ligand from the group shown in Table 1. Most preferably the bioconjugate comprises one or more peptides selected from the group consisting of P6-P16, P21-P30, P48-P104 and P109-P112 (Table 1).

In preferred methods for preventing and treating Crohn's disease (CD), there is administered an anti-CD-effective amount of bioconjugate comprising one or more peptides capable of binding selectively to target ligands expressed on cells in inflamed bowel tissue. Preferably, the bioconjugate comprises peptides capable of binding to the target ligand from the group shown in Table 1. Most preferably the bioconjugate comprises one or more peptides selected from the group consisting of P6-P16, P21-P30, P48-P104 and P109-112 (Table 1). The nucleotide sequences are provided in Table 2.

The utility of the compounds of the present invention as medical agents in the prevention and suppression of inflammatory cell responses to vulnerable tissue and as a therapeutic agent to prevent the pathological consequences of excessive inflammation in mammals (e.g., humans) is demonstrated by the activity of the compounds of this invention in cell adhesion assays described below in Examples 2 and 3.

Therapy of Disorders Due to Pathogenic Immune Responses

In a further aspect, the invention provides methods for treating or inhibiting a disorder due to pathogenic immune responses. Although leukocyte adhesion to tissue surfaces is essential for normal immune system function, leukocyte/tissue adhesion plays a major role in a number of pathological processes including septic shock, post-trauma multiple organ failure, ischemic reperfusion injury, transplant rejection, inflammatory diseases, and autoimmune diseases. Accordingly, these methods provide targeted therapeutics for these diseases.

Topical and systemic anti-inflammatory/immunosuppressant therapeutic methods are presented for treating and preventing leukocyte adhesion/infiltration, to suppress inflammation and to prevent the pathological processes that result from excess inflammation. Integrin-mediated leukocyte/tissue adhesion plays a major role in a number of these pathological processes.

Methods for treating and preventing ischemia-reperfusion injury are provided. In the methods an anti-ischemia-reperfusion-injury-effective amount of a bioconjugate comprising one or more peptides capable of binding selectively to target ligands expressed on endothelium is administered intravenously. In the methods the bioconjugate comprises peptides capable of binding to the target ligand. Most preferably the peptides may be selected from the group consisting of P6-P16, P21-P104 and P106-P112 (Table 1).

Therapy and Prevention of Infection By Pathological Agents

Methods are presented for preventing or treating pathogenic immune responses resulting from infection by bacteria, a biological warfare agent, anthrax or small pox, for example. Sexually transmitted diseases caused by bacterial pathogens or viral pathogens may likewise be prevented and treated. In these methods an effective immunosuppressive amount of a bioselective bioconjugate of the present invention is administered to an individual in need thereof.

Methods are presented for treatment of septic shock resulting from bacterial infection. Many bacteria (including agents of biological warfare, like anthrax) not only invade and infect host organisms, but also release endotoxins that promote a massive, systemic inflammatory response, resulting in an immune attack on healthy as well as diseased tissue. The present method protects tissues against injurious pathogenic immune responses. In certain instances the therapeutic method is used in adjunct with antibiotics to increase patient/casualty survival.

Infections of many types can result in hypersensitivity reactions, which are typically treated with steroids such as hydrocortisone and prednisolone, which have the drawback of side effects and interference with clearing the parasite (bacterial, viral or ameboid). Examples include SARS-related pulmonary hypersensitivity and hookworm infestation. In pulmonary infections, inflammatory exudates form in alveoli and bronchi and are organized by extensive matrix deposits and scarring. Ligands for integrins include CN III and CN IV.

Pancreatic infection results in damage to the ducts (epithelial cells), periductal inflammation, and new extracellular matrix expansion. Collagen also may be present and attract integrin-expressing cells.

In an important aspect, methods are presented for treatment of septic shock resulting from bacterial infection. Many bacteria (including agents of biological warfare like anthrax) not only invade and infect host organisms but also release endotoxins that promote a massive and systemic inflammatory response resulting in an immune attack on healthy as well as diseased tissue. Among the abnormalities is deposition of platelets on damaged epithelium. The present method protects tissues against injurious pathogenic immune responses. In certain instances the therapeutic method is used in adjunct with antibiotics to increase patient/casualty survival.

In methods for preventing and treating septic shock, an anti-septic shock effective amount of a bioconjugate comprising one or more peptides capable of binding selectively to integrin ligands expressed on endothelium. The product must be infused intravenously. Preferably, the bioconjugate comprises one or more peptides selected from the group consisting of P1-P16, P21-P30, P48-P102, P109-P110 (Table 1).

Therapy of Post-Trauma Multiple Organ Failure

Methods are presented to prevent and treat post trauma multiple organ failure. A bioselective bioconjugate of the present invention in a resuscitative fluid for preventing post-trauma multiple organ failure is presented.

Severe trauma can invoke a massive and systemic inflammatory response resulting in an immune attack on healthy as well as diseased tissue. The present methods may be used to protect tissues against injurious pathogenic immune responses that promote multiple organ failure. In this aspect, methods are presented for preventing the pathogenic results of intestinal ischemia and reperfusion that promote leukosequestration and injury in the gut as well as other organs resulting in multiple organ failure (MOF). Polymorphonuclear neutrophils (PMNs) play a key role in MOF since they respond to injury by adhering to tissues in multiple organs and releasing injurious oxidative agents.

In methods for preventing and treating multiple organ failure (MOF), an anti-MOF-effective amount of a bioconjugate comprising one or more peptides capable of binding selectively to target ligands expressed on endothelial cells. Preferably the bioconjugate comprises one or more peptides selected from the group consisting of P1-16, P21-104 and P106-P112 (Table 1).

Treatment of Wound Trauma

Means are presented for preventing and treating individuals suffering from severe trauma and injuries. Although massive blood loss and dehydration are the primary life-threatening factors in trauma patients, a major downstream effect of these severe injuries is a massive arousal of the immune system. Unfortunately this immune response is so aggressive that healthy tissues are destroyed by immune system cells (typically white blood cells) attempting to clean up and eliminate dead, injured tissues. This collateral damage of healthy tissue can promote failure of healthy organs and decrease patient survivability. The present bioconjugates may be used in intravenous replacement fluids, such as Ringer's lactate, where they circulate in the blood and selectively form a barrier on the endothelium to prevent attack by PMNs. Preferably the bioconjugates are incorporated into a formulation that replaces fluid loss to curtail collateral damage to healthy tissues that inevitably occurs following severe injuries. In these embodiments, the bioselective bioconjugates may be incorporated into blood replacements that are shipped in a dry or lyophilized formulation in conventional fluid therapy bags or are otherwise added to the conventional intravenous fluids.

Targeted and localized protection from pathogenic immune responses triggered by diseases that cause ischemic injury (injury due to lack of oxygen), e.g., heart attack and stroke, are also presented.

Prevention of Transplant Rejection

In another aspect of the invention, methods are presented for locally suppressing transplant rejection of allograft organ transplants including heart, lung, liver, kidney, skin, pancreatic islets, and cornea. In these methods biospecific bioconjugates target ICAM-1 on organ transplants, reducing or eliminating inflammation and the need for traditional systemic immunosuppression therapy, which is less specific.

Prevention and Treatment of Autoimmune Disease.

Also presented are targeted and localized methods for protection from autoimmune diseases, including, but not limited to, diabetes and rheumatoid arthritis. At least ICAM-1 and LFA-1 are implicated in autoimmune diseases. Blocking those receptors is a strategy for blocking autoimmune reactions and limiting conditions such as diabetes and rheumatoid arthritis. MAdCAM-1 receptors also have been implicated in diabetes.

Prevention of Atherosclerosis

Atherosclerosis is an inflammatory condition. Endothelium is injured by a variety of sources (elevated cholesterol, hypertension, etc.) and begins to display receptors that are ligands for integrins. The receptors include but are not limited to ICAM-1, VCAM-1 (vascular cell adhesion molecule) and PDGF.

Treatment and Prevention of Cirrhosis

Cirrhosis is the replacement of hepatocytes with fibrotic cells and is due to an inflammatory processes such as hepatitis and toxic reactions. Ligands for integrins also are present in cirrhosis. These include collagen I and III (CN I and CN III).

Treatment and Preventions of Glomerulosclerosis

This disorder is characterized by inflammatory destruction of renal glomeruli and replacement by fibrotic scar tissue. Such pathology is associated with the presence of CN I, CN IV and fibrinogen, which serve as ligands for integrins.

Prevention of Cancer Metastasis

Tumor metastasis is a fine-tuned balance between the formation and loosening of adhesive cell contacts within the tumor, which is regulated by various integrins. For example, human ovarian cancer cells express integrin αᵥβ₃, which associates with vitronectin in the extracellular matrix and correlates with cancer progression. Exposure of such cancer cells to vitronectin results in proliferation and motility increase of five fold. Once blood-borne metastatic cancer cells may lodge in the lungs, causing early, intravascular metastatic tumors. Pulmonary vasculature contains integrin ligands known as calcium-activated chloride channels (CLCA) which are specific for the specific-determining loop (SDL) of β₄. Two mechanisms of fighting cancer metastasis are blocking vitronectin with the ligand-binding portion of αᵥβ₃ and blocking the CLCA ligand with a peptide including amino acids (SEQ ID NOS 184-203) of integrin β₄.

Sequelae of Viper and Rattlesnake Bites

Snake bites may cause excessive capillary permeability, which may be mediated by integrins.

### EXAMPLES

### Example 1

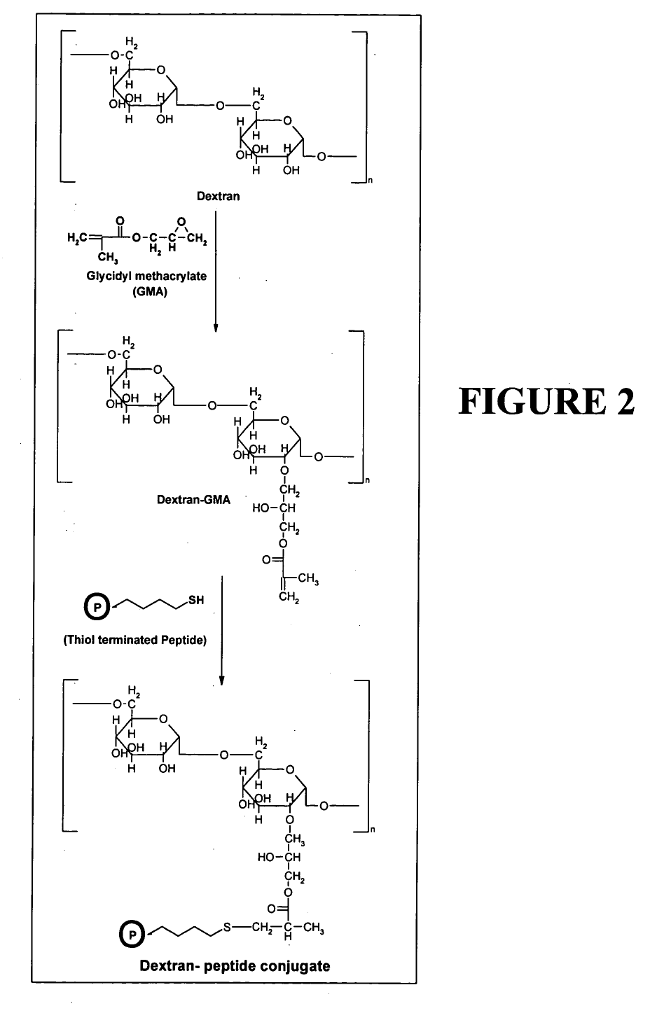

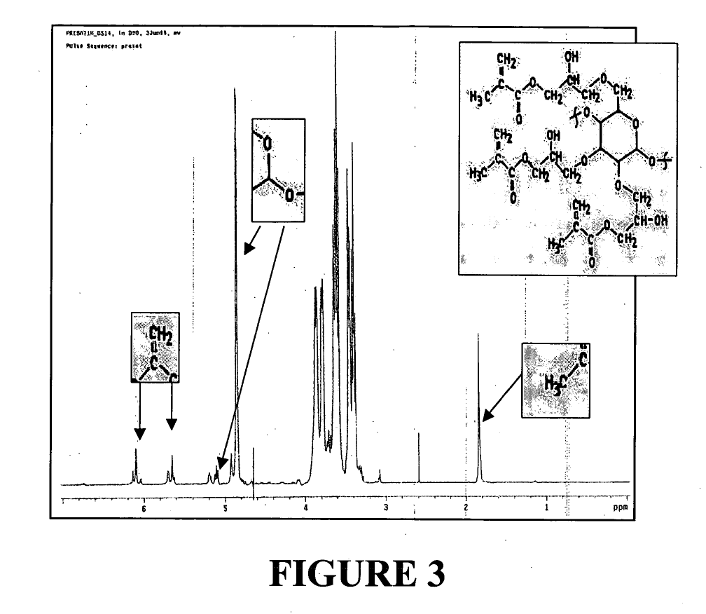

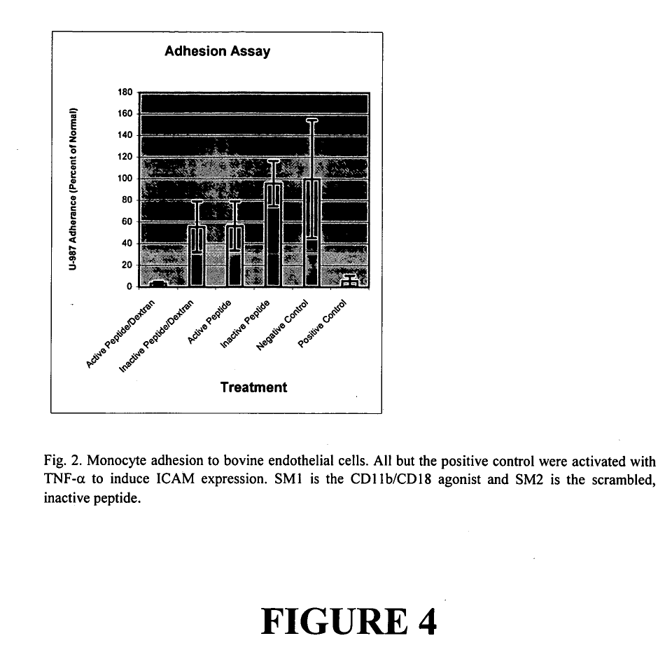

This experiment presents the synthesis of a preferred embodiment of the present invention, an anti-inflammatory dextran/peptide bioconjugate. This reaction scheme is illustrated in FIG. 2.

Synthesis and Chemical Characterization of Methacroylated Dextran

Dextran, molecular weight about 70 kD (25 g), and dimethylaminopyridinine (DMAP) (5 g) were dissolved in dimethylsulfoxide (DMSO) (225 ml) under nitrogen atmosphere at room temperature. Glycidyl methacrylate (GMA), a linking molecule, was added to the mixture to produce GMA-derivatized dextran (dex-GMA). The amount of GMA was adjusted to obtain 10 degrees of substitution (DS) (DS: molar ratio of GMA per glucopyranose residue). The reaction was terminated after 48 hours. The product was purified from the reaction mixture by solvent removal and size exclusion chromatography. Aqueous solutions of methacroylated dextran were rapidly frozen in liquid nitrogen, lyophilized, and stored frozen. FIG. 2 illustrates the chemical structures of dextran, GMA, and methacroylated dextran and the dextran-peptide bioconjugate. FIG. 3 is an NMR of dextran.

Synthesis of the Anti-Inflammatory Dextran/Peptide Bioconjugate by Coupling a Synthetic Peptide (CNAFKILVVITDGEK) to Activated Dextran

The synthetic peptide was based on the portion of integrin αₘβ₂ (CD11b/CD18) that fits in the ICAM-1-binding pocket. Synthesis with this peptide is illustrative and other peptides may likewise be coupled to dextran or other polyvalent polymers. The synthetic peptide (CNAFKILVVITDGEK) was added to phosphate buffered saline (PBS) with 1.5 mM EDTA at a final concentration of 20 mM. The pH was adjusted to 8.0-8.5 with triethanolamnine (TEA). Methacroylated dextran (2 mM) was then added to the reaction mixture and the pH was adjusted again to pH 8.0-8.5 with TEA. All solutions were maintained under inert conditions to minimize disulfide bond formation. Crosslinking was allowed to proceed at room temperature for two hours. The reaction mixture was then dialyzed against deionized water in 25,000 MWCO membrane to remove any unreacted or disulfide-bonded peptide. The purified dextran/peptide conjugates were recovered by lyophilization.

A bioconjugate containing an inactive scrambled sequence of the above A-domain peptide CTVDLKFGIKNIEAV, was similarly synthesized and was conjugated to dextran and used as the sham control in the in vitro assays described below. Synthetic peptides were added to phosphate buffered saline (PBS) with 1.5 mM EDTA at a final concentration of 20 mM. The pH was adjusted to 8.0-8.5 with TEA. Methacroylated dextran (2 mM) was then added to the reaction mix and the pH was adjusted again to pH 8.0-8.5 with TEA. All solutions were maintained under inert conditions to minimize disulfide bond formation. Crosslinking was allowed to proceed at room temperature for two hours. The reaction mixture was then dialyzed against deionized water in 25,000 MWCO membrane to remove any unreacted or disulfide-bonded peptide. The purified dextran/peptide conjugates were recovered by lyophilization.

### Example 2

This experiment illustrates the activity of the bioconjugate, whose synthesis was described above, in the inflammatory cell adhesion assay. Bovine endothelial cell (BEC) monolayers were established in 24-well culture dishes. At 24 h prior to the assay, normal medium (Minimal Eagle's Medium with 10% fetal bovine serum, 1% ABAM and 1% L-glutamine) (Gibco, CA, USA) was replaced with medium containing tumor necrosis factor α (TNF-α, 10 ng/ml). Following the 24 h incubation period, each sample well received a medium change.

Treated sample groups received medium containing 6% dextran bioconjugate or 6% bioconjugate. Negative control samples received medium containing dextran bioconjugate whose peptide had a scrambled A domain sequence. Two other control treatments were given: a medium change with no dextran or peptide was given to a sample group pretreated with TNF-α, and a positive control that was not pretreated with TNF-α. After a 30-minute incubation period, the medium in all wells was replaced with medium containing the human monocyte cell line U937 (1×10⁵/ml) (ATCC, Manassas, Va.). All samples were incubated for another 30 minutes, then washed three times with PBS to remove non-adherent cells. The average number of adherent cells per 100× microscopic field was determined for each sample group.

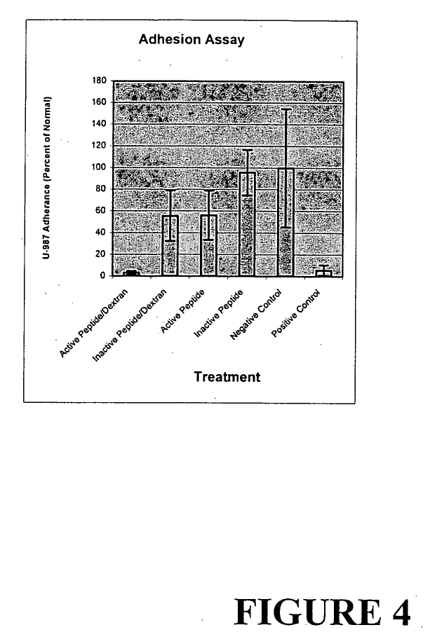

Referring to FIG. 4, the results of this assay illustrate the biospecific binding of the peptide/dextran conjugate to bovine endothelial cells. In this assay all but the positive control were activated with TNF-α to induce ICAM expression. The negative control represents 100%. Treatment with active peptide conjugate resulted in a relative monocyte adherence of 3.34±1.69%. The positive control, where the endothelial cells were not induced, had monocyte adherence of 5.741±4.81%, which is not statistically different from samples where ICAM expression was induced preceding treatment with the active conjugate. The treatment with the inactive peptide conjugate yielded a relative adherence of 55.65±23.42%, while treatment with the active peptide alone led to a monocyte adherence of 56.28±22.67%. The treatment with the inactive peptide alone was comparable to no treatment after the TNF-α activation. Inactive peptide treatment gave a relative monocyte adherence of 95.71±21.03%. The standard deviation for the negative control was 54.5.

The active dextran bioconjugate effectively bound to TNF-α stimulated, ICAM-expressing BECs and prevented monocyte adhesion to the extent observed in non-stimulated BECS (positive control). Unconjugated peptides, dextran, and the inactive peptide conjugate inhibited cell adhesion poorly, suggesting that only the combined effect of specific binding of active peptide conjugates to ICAM and formation of an ICAM-bound nonadhesive dextran layer promoted reduced monocyte adhesion to TNF-α stimulated, ICAM-expressing BECs. Since leukocyte/tissue adhesion plays a major role in a number of the pathological processes discussed above, these bioconjugates could be utilized as targeted therapeutics for many applications.

### Example 3

This experiment illustrates the inhibition of leukocyte/inflamed cell binding in human umbilical vein endothelial cell (HUVEC) monolayers by the bioselective bioconjugates of the present invention.

To assess the effect of these peptide-dextran bioconjugates on inflammatory cell adhesion, the following in vitro ICAM-1-mediated leukocyte cell adhesion assay was performed. HUVEC monolayers were established in 24-well culture dishes. At 24 h prior to the assay, normal culture media were replaced with medium containing TNF-α (10 ng/ml). Following the 24 h incubation period, each sample well received a medium change. Treated sample groups received medium containing 6% dextran bioconjugate (dextran conjugated to the A domain peptide CNAFKILVVITDGEK). Untreated control samples received normal medium. Negative sham control samples received medium containing dextran conjugate with a scrambled A domain sequence (KCENGADFTKIIVLV). All samples were then incubated for 30 min prior to the adhesion assay. Medium was removed from all wells following the 30 min incubation and replaced with medium containing U937 monocytic cells (1×10⁵/ml). All samples were then incubated for another 30 min. After this incubation period, samples were washed three times with PBS to remove non-adherent monocytes. The samples were then fixed, and an average number of adherent monocytes per 100× microscopic field was determined for each sample group. Statistical comparisons between sample groups (n=4 replicate wells per group) were performed using a student's t-test.

U937 cell adhesion to inflammatory HUVECs was reduced by 87.7% in the sample group treated with bioconjugate containing the active A-domain sequence CNAFKILVVITDGEK. No significant reductions in cell adhesion were observed in untreated and sham-treated (scrambled A domain peptide conjugated to dextran) sample groups.

It should be understood that the invention is not limited to the particular embodiments described herein, but that various changes and modifications may be made without departing from the spirit and scope of this novel concept as defined by the following claims. The following references are incorporated by reference.

References

1 D S Tuckwell, L Smith, M Korda, J A Askari, S Santosof, M J Barnes, R W Farndale, and M J Humphries, Monoclonal antibodies identify residues 199-216 of the integrin α₂ vWFA domain as a functionally important region within α₂/β₁. Biochem J (2000) 350: 485-493.

2 S L King, T Kamata, J A Cunningham, J Emsley, R C Liddington, Y Takada, and J M Bergelson, Echovirus-1 interaction with the human very late antigen-2 (integrin α₂/β₁) I domain. J Biol Chem (1997) 272: 285518-28522.

3 T Kamata, R C Liddington, and Y Takada, Interaction between collagen and α₂ I domain of integrin α₂/β₁. J Biol Chem (1999) 274: 32108-32111.

4 T Kamata, W Puzon, and Y Takada, Identification of putative ligand binding sites within I domain of integrin α₂/β₁(VLA-2, CD49b/CD29). J Biol Chem (1994) 269: 9659-9663.

5 S G Schiffer, M E Hemler, R R Lobb, R Tizard, and L Osborn, Molecular mapping of functional antibody binding sites of α₄ integrin. J Biol Chem (1995) 270: 14270-14273.

6 A Irie, T Kamata, and Y Takada, Multiple loop structures critical for ligand binding of the integrin α₄ subunit in the upper face of the beta-propeller mode 1. Proc Natl Acad Sci USA 1997; 94: 7198-7203.

7 A Irie, T Kamata, W Puzon-McLaughlin, and Y Takada, Critical amino acid residues for ligand-binding are clustered in a predicted beta-turn of the 3^{rd} N-terminal repeat in the integrin α₄ and α₅ subunits. EMBO J (1995) 14: 5550-5556.

8 T Kamata, W Puzon, and Y Takada, Identification of putative ligand-binding sites within of the integrin α₄β₁ (VLA-2, CD49d/CD29). Biochem J (1995) 305: 945-951.

9 Z Cao, K Huang, and A F Horwitz, Identification of a domain on the integrin α₅ subunit implicated in cell spreading and signaling. J Biol Chem (1998) 273: 31670-31679.

10 J L Baneres, F Roquet, M Green, H LeCalvez, and J Parello, The cation-binding domain from the alpha subunit of integrin α₅β₁ is a minimal domain for fibronectin recognition. J Biol Chem (1998) 273: 24744-24753.

11 A P Mould, J Askari, Humphries M J, Molecular basis of ligand recognition by integrin α₅β₁. J Biol Chem (2000) 275: 20324-20336.

12 U.S. Pat. No. 5,843,885, Benedict et al (1998)

13 Yusuf-Makagiansar H, Siahaan T J. Binding and internalization of an LFA-1-derived cyclic peptide by ICAM receptors on activated lymphocyte: A potential ligand for drug targeting to ICAM-1 expressing cells. Pharm Res 2001:18:329-335.

14 Yusuf-Makagiansar H, Makagiansar I T, Siahaan T J. Inhibition of the adherence of T-lymphocytes to epithelial cells by a cyclic peptide derived from inserted domain of lymphocyte function-associated antigen-1. Inflammation 2001:25:203-214.

15 Jois S D, Tibbetts S A, Chan M A, Benedict S H, Siahaan T J. A Ca²⁺ binding cyclic peptide derived from the a-subunit of LFA-1: Inhibitor of ICAM-1/LFA-1-mediated T-cell adhesion. J Pept Res 1999:53:18-29.

16 T Kamata, K K Tieu, T Tarui, W Puzon-McLaughlin, N Hogg, and Y Takada. The role of CPNKEKEC sequence in the beta 2 subunit I domain in regulation of integrin α_{L}β₂ (LFA-1). (2002) J Immunol 168: 2296-2301.

17 L Zhang and E Plow, Amino acid sequences within the alpha subunit of integrin αₘβ₂ (Mac-1) critical for specific recognition of C3bi. Biochem (1999) 38: 8064-8071.

18 V P Yakubenko, D A Solovjov, L Zhang, V C Yee, E F Plow, and T P Ugarova. Identification of the binding site for fibrinogen recognition peptide gamma 383-395 within the alpha m I-domain of integrin αₘβ₂ (2001) 276: 13995-14003.

19 J Plescia, M S Conte, G VanMeter, G Ambrosini, and D C Altieri. Molecular identification of the cross-reacting epitope on α_{b}β₂ integrin I domain recognized by anti-α_{IIb}β₃ monoclonal antibody 7E3 and its involvement in leukocyte adherence. J Biol Chem (1998) 273: 20372-20377.

20 J J Calvete, W Schafer, K Mann, A Henschen, and J Gonzalez-Rodriguez. Localisation of the cross-linking sites of RGD and KQAGDV peptides to the isolated fibrinogen receptor, the human platelet integrin glycoprotein IIb/IIIa-Influence of peptide length. (1992) Eur J Biochem 206: 759-765.

21 J J Calvete, G Rivas, W Schafer, M A McLane, and S Niewiarowski. Glycoprotein IIb peptide 656-667 mimics the fibrinogen gamma chain 402-411 binding site on platelet integrin GPIIb/IIIa (1993) FEBS Lett 235: 132-135.

22 D B Taylor, J M Derrick, and T K Gartner. Antibodies to GPIIb (alpha)(300-312) inhibit FG binding, clot retraction, and platelet adhesion to multiple ligands (1994) Proc Soc Exp Biol Med 205: 35-43.

23 J M Grunkemeier and T A Horbett. Fibrinogen receptor-like biomaterials made by pre-adsorbing peptides to polystyrene substrates (1996) J Mol Recog 9: 247-257.

24 L J Yao and K H Mayo. Interactions of integrin GPIIb/IIIa-derived peptides with fibrinogen investigated by NMR spectroscopy (1996) Biochem J 315: 161-170.

25 E M Makogonenko, V P Yakubenko, K C Ingham, and L V Medved. Thermal stability of individual domains in platelet glycoprotein IIbIlla (1996) Eur J Biochem 237: 205-211.

26 S E D'Souza, M H Ginsberg, T A Burke, and E F Plow. The ligand binding site of the platelet integrin receptor GPIIb-IIIa is proximal to the second calcium binding domain of its alpha subunit (1990) J Biol Chem 265: 3440-3446.

27 D Gulino, C Boudignon, L Zhang, E Concord, M J Rabiet, and G Maguerie. Calcium-binding properties of the platelet glycoprotein IIb ligand-interacting domain (1992) J Biol Chem 267: 1001-1007.

28 W Puzon-McLaughlin, T Kamata, and Y Takada. Multiple discontinuous ligand-mimetic antibody binding sites define a ligand binding pocket in integrin α_{IIb}β₃. (2000) J Biol Chem 275: 7795-7802.

29 Y K Liu, A Nemoto, Y Feng, T Uemura. The binding ability of matrix proteins and the inhibitory effects on cell adhesion of synthetic peptides derived from a conserved sequence of integrins. (1997) J Biochem 121: 961-968.

30 G Bazzoni, D T Shih, C A Buck, and M E Hemler. Monoclonal antibody 9EG7 defines a novel β₁ integrin epitope induced by soluble ligand and manganese, but inhibited by calcium. (1995) J Biol Chem 270: 25570-25577.

31 J Takagi, Kamata T, J Meredith and W Puzon-McLaughim. Changing ligand specificities of αᵥβ₁ and αᵥβ₃ integrins by swapping a short diverse sequence of the beta subunit. (1997) J Biol Chem 272: 19794-19800.

32 W Puzon-McLaughlin and Y Takada. Critical residues for ligand binding in an I domain-like structure of the integrin β₁ subunit. (1996) J Biol Chem 271: 20438-20443.

33 Y Takada and W Puzon. Identification of regulatory region of integrin β₁ subunit using activating and inhibiting antibodies. (1993) J Biol Chem 268: 17597-17601.

34 H Y Ni and J A Wilkins. Localisation of a novel adhesion blocking epitope on the human beta 1 integrin chain. (1998) Cell Adhesion and Comm 5: 257-271.

35 L L Chen, R R Lobb, J H Cuervo, K C Lin, S P Adams, and E B Pepinsky. Identification of ligand binding sites on integrin α₄β₁ through chemical cross-linking. (1998) 37: 8743-8753.

36 D T Shih, D Boettiger, and C A Buck. Epitopes of adhesion-perturbing antibodies map within a predicted alpha helical domain of the integrin β₁ subunit. (1997) 110: 2619-2628.

37 C Huang, Q Zang, J Takagi, and T A Springer. Structural and functional studies with antibodies to the integrin β₂ subunit. (2000) 275: 21514-21524.

38 Y M Xiong and L Zhang. Structure-function of the putative I-domain within the integrin β₂ subunit. (2001) 276: 19340-19349.

39 Y M Xiong, T A Haas, and L Zhang. Identification of functional segments within the β₂ I-domain of integrin αₘβ₂. (2002) 277: 46639-46644.

40 C F Lu, M Ferzly, J Tagaki, and Springer T A. Epitope mapping of antibodies to the C-terminal region of the integrin β₂ subunit reveals regions that become exposed upon receptor activation. (2001) 166: 5629-5637.

41 P Rieu, T Ueda, I Haruta, C P Sharma, and M A Amaout, The A domain of β₂ integrin CR3 (CD11b/CD18) is a receptor for the hookworm-derived neutrophils adhesion inhibitor NIF. J Cell Biol 1994; 127: 2081-2091.

42 Tibbetts S A, Chirathawom C, Nakashima M, Jois S D S, Siahaan T J, Chan M A, Benedict S H. Peptides derived from ICAM-1 and LFA-1 modulate T cell adhesion and immune function in a mixed lymphocyte culture. Transplantation 1999:68:685-692.

43 Jois S D S, Hughes R, Siahaan T J. Comparison of solution conformations of a cell-adhesive peptide LBE and its reverse sequence EBL. J Biomol Struc Dyn 1999:17:429-444.

44 S E D'Souza, M H Ginsberg, T A Burke, S C T Lam, and E F Plow. Localization of an Arg-Gly-Asp recognition site within and integrin adhesion receptor. Science (1990) 242: 91-93.

45 J J Cook, M Trybulec, E C Lasz, S Khan, and S Niewiaeowski. Binding of glycoprotein-IIIa-derived peptide 217-231 to fibrinogen and von Willebrand factor and its inhibition by platelet glycoprotein IIb/IIIa complex. (1992) Biochim Biophys Acta 1119: 312-321.

46 J J Calvete, K Mann, M V Alvarez, M M Lopez, and J Gonzales-Rodriguez. Proteolytic dissection of the isolated platelet fibrinogen receptor, integrin gp IIb/IIIa-localization of gpIIb and gp IIIa putatively involved in the subunit interface and in intrasubunit and intrachain contacts. (1992) Biochem J 282: 523-532.

47 M L Bajt, M H Ginsberg, A L Frelinger, M C Berndt, and J C Loftus. A spontaneous mutation of integrin α_{IIb}β₃ (platelet glycoprotein IIb-IIIa) helps define a binding site. (1992) J Biol Chem 267: 3789-3794.

48 B Steiner, A Trzeciak, G Pfenninger, and W C Kouns. Peptides derived from a sequence within β₃ integrin bind to a platelet α_{IIb}β₃ (gpIIb-IIIa) and inhibit ligand binding. (1993) J Biol Chem 268: 6870-6873.

49 E C Lasz, M A McLane, M Trybulec, M A Kowalska, S Khan, A Z Budzynski, and S Niewiarowski. β₃ integrin derived peptide 217-230 inhibits fibrinogen binding and platelet aggregation: significance of RGD sequences and fibrinogen A alpha chain. (1993) Biochem Biophys Res Comm 190: 118-124.

50 J J Calvete, M A McLane, G J Stewart, and S Niewiarowski. Characterization of the cross-linking site of disintegrins albolabrin, bitistatin, echistatin, and eristostatin on isolated human platelet integrin gpIIb/IIIa. (1994) Biochem Biophys Res Comm 202: 135-140.

51 S E D'Souza, T A Haas, R S Piotrowicz, V Byersward, D E McGrath, H R Soule, C Chemiewski, E F Plow, J W Smith. Ligand and Cation-binding are dual functions of a discrete segment of the integrin β₃ subunit—cation displacement is involved in ligand-binding. (1994) Cell 79: 659-667.

52 S Honda, Y Tomiyama, A J Pelletier, D Annis, Y Honda, R Oechekowski, Z Ruggeri, and T J Kunicki. Topography of ligand-induced binding sites, including a novel cation-sensitive epitope (AP5) at the amino terminus, of the human integrin β₃ subunit. (1995) J Biol Chem 270: 11947-11954.

53 W C Kouns, P J Newman, K J Puckett, A A Miller, C D Wall, C F Fox, J M Seyer, and Lennings L K. Further characterization of the loop structure of platelet glycoprotein IIIa-partial mapping of functionally significant glycoprotein IIIa epitopes. (1991) Blood 78: 3215-3223.

54 X Du, M Gu, J W Weisel, C Nagaswami, J S Bennett, R Bowditch, and M H Ginsberg. Long range propagation of conformational changes in integrin α_{IIb}β₃. J Biol Chem (1993) 268: 23087-23092.

55 R Pasqualini, E Koivunen, and E Ruoslahti. A peptide isolated from phage display libraries is a structural and functional mimic of an RGD-binding site on integrins. (1995) J Cell Biol 130: 1189-1196.

56 M Alemany, E Concord, J Garin, M Vincon, A Giles, G Marguerie, and D Gulino. Sequence 274-368 in the β₃ subunit of the integrin α_{IIb} β₃ provides a ligand recognition and binding domain for the gamma chain of fibrinogen that is independent of platelet activation. Blood 87: 592-601.

57 E C K Lin, B I Ratnikov, P M Tsai, C P Carron, D M Myers, C F Barbas, and J W Smith. Identification of a region in the integrin β₃ subunit that confers ligand binding specificity. (1997) J Biol Chem 272: 23912-23920.

58 I Wierzbicka, M A Kowalska, E C Lasz, D H Farrell, A Z Budzynski, and S Niewiarowski. Interaction of β₃ integrin-derived peptides 214-218 and 217-231 with α_{IIb}β₃ complex and with fibrinogen A alpha chain (1997) Thromb Res 85: 115-126.

59 M Triantafilou, K Triantafilou, K M Wilson, Y Takada, and N Fernandez. High affinity interactions of coxsackievirus A9 with integrin αᵥβ₃ (CD51/61) require the CYDMKTTC sequence of β₃, but do not require the RGD sequence of the CAV-9 VP-1 protein. (2000) Human Immnol 61: 453-459.

60 G Bitan, L Scheibler, Z Greenberg, M Rosenblatt, and M Chorev. Mapping of the integrin αᵥβ₃-ligand interface by photoaffinity cross-linking. Biochem (1999) 38: 3414-3420.

61 G Bitan, L Scheibler, D F Mierke, M Rosenblatt, and M Chorev. Ligand-integrin αᵥβ₃ interaction determined by photoaffinity cross-linking. Biochem (2000) 39: 11014-11023.

62 L Scheibler, D F Mierke, G Bitan, M Rosenblatt, and M Chorev. Identification of a contact domain between echistatin and the integrin αᵥβ₃ by photoaffinity cross-linking. Biochem (2001) 40: 15117-15126.

63 A Ciemiewska-Cieslak, C S Cierniewska, K Blecka, M Paperak, L Michalec, L Zhang, and E F Plow. Identification and characterization of two cation binding sites in the integrin β₃ subunit. J Biol Chem (2002) 277: 11126-11134.

64 M Tidswell, R Pachynski, S W Wu, S Q Qiu, E Dunham, N Cochran, M J Briskin, P J Kilshaw, A I Lazarovitis, D P Andrew, Butcher E C, Yednock T A, and Earle D J. Structure-function analysis of the integrin β₇ subunit: identification of domains involved in adhesion to MAdCAM-1. J Immunol (1997) 159: 1497-1505.

65 T K Gartner and D B Taylor. The peptide Glu-His-Ile-Pro-Ala binds fibrinogen and inhibits platelet aggregation and adhesion to fibrinogen and vitronectin. (1991) Proc Soc Exp Biol Med 198: 649-655.

66 V Castronovo, G Tarboletti, and M E Sobel. Laminin receptor complementary DNA-deduced synthetic peptide inhibits cancer cell attachment to endothelium. (1991) Canc Res 51: 5672-5678.

67 S E D'Souza, V J Byers-Ward, E E Gardiner, H Wang, and S S Sung. Identification of an active sequence within the first immunoglobulin domain of intercellular molecule-1 (ICAM-1) that interacts with fibrinogen (1996) J Biol Chem 271: 24270-24277.

68 J P Shannon, M V Silva, D C Brown, and R S Larson. Novel cyclic peptide inhibits intercellular adhesion molecule-1 mediated cell aggregation. (2001) J Peptide Res 58: 140-150.

69 Kam J L, Regimbald L H, Hilgers J H M, Hoffman P, Krantz M J, Longenecker B M, Hugh J C. MUC1 synthetic peptide inhibition of intercellular adhesion molecule-1 and MUC1 binding requires six tandem repeats. (1998) Canc Res 58: 5577-5581.

70 J K Welply, C N Steininger, M Caparon, M L Michener, S C Howard, L E Pegg, D M Meyer, De Ciechi, C S Devine, G F Casperson. A peptide isolated by phage display binds to ICAM-1 inhibits binding to LFA-1. (1996) Proteins Struct Funct Genetics 26: 262-270.

We claim:

1. A therapeutic bioconjugate comprising:
a. a hydrophilic polymer; and
b. one or more peptides capable of binding specifically to a ligand expressed on a cell surface.

2. The bioconjugate of claim 1 for blocking interactions between cells in a living tissue wherein said ligand is expressed on the surface of at least one of said cells.

3. The bioconjugate of claim 1 for blocking interaction between a cell and an extracellular matrix wherein said ligand is capable of binding to a component of said matrix.

4. The bioconjugate of claim 1 for blocking pathological reactions triggered by cellular interactions in a living tissue.

5. The bioconjugate of claim 1 wherein said peptide comprises the amino acid sequence of the binding portion of an integrin for said ligand.

6. The bioconjugate of claim 5 for blocking cell signaling receptors implicated in the regulation of cellular adhesion, migration, tumor metastasis, proliferation, angiogenesis, bone resorption, apoptosis, or gene expression.

7. The bioconjugate of claim 5 wherein said binding portion is from an integrin α subunit or an integrin β subunit.

8. The bioconjugate of claim 7 comprising one or more peptides selected from the group consisting of SEQ ID NOS 1-202.

9. The bioconjugate of claim 7 wherein said binding portion is a portion of the integrin α₂ subunit (CD49b, VLA-2, platelet gpla) I domain, integrin α₄ (CD49b, VLA-4), integrin α₅ (CD49e, VLA-5) integrin α_{L} (CD11a) I domain, integrin α_{M} subunit (CD11b) I domain, integrin α_{I1b} I domain, integrin α_{IIb} (CD41) heavy chain, integrin α_{11b} (CD41) light chain, integrin β₁ (CD29) subunit, the integrin β₂ (CD18) subunit, integrin β₃ (CD61) subunit, or integrin β₇ (LPAM-1) subunit.

10. The bioconjugate of claim 9 wherein said peptide comprises the binding portion of the integrin α₂ subunit (CD49b, VLA-2, platelet gpla) I domain and binds specifically to ligands CN I, CN II, CN III, CN IV, LN or the echovirus-1 receptor.

11. The bioconjugate of claim 9 wherein said peptide comprises a portion of the integrin α₄ (CD49b, VLA-4) subunit that binds specifically to the ligands VCAM-1, FN, MAdCAM-1, TSP or invasin.

12. The bioconjugate of claim 9 wherein said peptide comprises a portion of the integrin α₅ (CD49e, VLA-5) that binds specifically to ligands FN, L1 or invasin.

13. The bioconjugate of claim 9 wherein said peptide comprises a portion of the integrin α₁ (CD11a) I domain that binds specifically to the ligands ICAM-1, ICAM-2, ICAM-3 or LPS.

14. The bioconjugate of claim 9 wherein said peptide comprises a portion of the integrin α_{M} subunit (CD11b) I domain that binds specifically to the ligands iC3b, ICAM-1, ICAM-2, ICAM-4, Fb, Factor X, CD23, NIF, heparin, beta glucan, or LPS.

15. The bioconjugate of claim 9 wherein said peptide comprises a portion of the integrin α_{I1b} (CD41) heavy chain that binds specifically to the ligands Fb, FN, VN, TSP or vWF.

16. The bioconjugate of claim 9 wherein said peptide comprises a portion of the integrin α_{11b} (CD41) light chain that binds specifically to the ligands Fb, FN, VN, TSP and vWF.

17. The bioconjugate of claim 9 wherein said peptide comprises a portion of the integrin β₁ (CD29) subunit, and binds specifically to the ligands FN, LN, CN, VCAM-1, FN, MAdCAM-1, TSP or invasin.

18. The bioconjugate of claim 9 wherein said peptide comprises a portion of the integrin β₂ (CD18) subunit that binds specifically to the ligands ICAM-1, ICAM-2, ICAM-3, ICAM-4, LPS, iC3b, Fb, Factor X, CD23, NIF, heparin, or betaglucan.

19. The bioconjugate of claim 9 wherein said peptide comprises a portion of the integrin β₃ (CD61) subunit that binds specifically to ligands fibrinogen, fibronectin, vitronectin, thrombospondin, von Willebrand factor, osteopontin, bone sialoprotein, laminins, collagens, or neural cell adhesion molecule L1.

20. The bioconjugate of claim 9 wherein said peptide comprises a portion of the integrin β₇ (LPAM-1) subunit that binds specifically to the ligands VCAM-1, fibronectin, MAdCAM-1, or E-cadherin (cadherin-1).

21. The nucleic acids having the sequence coding for peptides of the bioconjugate of claim 8.

22. The nucleic acids of claim 21 selected from the group consisting of SEQ ID NOS 1, 3, 5, 7,9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 86, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 186, 185, 187, 189, 191, 193, 195, 1197, 199 and 201.

23. The peptide for preparation of the bioconjugate of claim 1, said peptide having a sequence selected from the group consisting of SEQ ID NOS 1-112, wherein each sequence comprises additionally an N-terminal and/or a C-terminal cysteine residue.

24. The nucleic acids having the sequence coding for a peptide of claim 23.

25. The bioconjugate of claim 1 wherein said polymer is a polysaccharide or an oligosaccharide.

26. The bioconjugate of claim 1 wherein said polymer is a derivative of a polysaccharide or an oligosaccharide wherein said derivative polymer additionally comprises additional groups capable of reacting chemically with a peptide to form said bioconjugate.

27. The bioconjugate of claim 1 having the formula XY_{b} wherein X is a low cell-adhesive, hydrophilic polymer, Y is a peptide comprising a portion of the binding site of an integrin for a ligand expressed on a cell surface, and b is greater than 0.

28. The bioconjugate of claim 27 wherein X comprises a polysaccharide or an oligosaccharide.

29. The bioconjugate of claim 27 wherein X comprises a derivative of a polysaccharide or of an oligosaccharide wherein said derivative saccharide comprises reactive groups whereby said derivative saccharide reacts with said peptide to form said bioconjugate.

30. The bioconjugate of claim 29 wherein said reactive group comprises a hydroxyl group.

31. The bioconjugate of claim 25 wherein said polysaccharide or oligosaccharide is selected from the group consisting of agarose, dextran, heparin, chondroitin sulfate, hydroxyethyl starch, and hyaluronic acid.

32. The bioconjugate of claim 1 wherein said polymer comprises a dextran and said peptide comprises the binding portion of an integrin for its ligand.

33. The bioconjugate of claim 1 wherein said polymer is polyvalent and is selected from the group consisting of poly(ethylene glycol), poly(ethylene oxide), poly(vinyl alcohol), poly(acrylic acid), poly(ethylene-co-vinyl alcohol), poly(vinyl pyrrolidone), poly(ethyloxazoline), and poly(ethylene oxide)-co-poly(propylene oxide) block copolymers.

34. The bioconjugate of claim 1 wherein said polymer comprises copolymers, block copolymers, graft copolymers, alternating copolymers, or random copolymers.

35. The bioconjugate of claim 1 wherein said polymer is essentially inert.

36. The bioconjugate of claim 1 wherein said polymer is degradable by hydrolytic or enzymatic means.

37. The bioconjugate of claim 36 wherein said degradable polymer comprises one or more blocks selected from the group consisting of lactic acid, glycolic acid, 6-caprolactone, lactic-co-glycolic acid oligomers, trimethylene carbonate, anhydrides, and amino acids.

38. The bioconjugate of claim 1 wherein said polymer is a serum protein.

39. The bioconjugate of claim 38 wherein said serum protein is an albumin.

40. The bioconjugate of claim 1 in a pharmaceutically acceptable carrier.

41. The bioconjugate of claim 1 immobilized on a solid substrate.

42. The bioconjugate of claim 41 wherein said substrate is an implantable medical device.

43. The bioconjugate of claim 42 wherein said medical device is a drug delivery device.

44. The bioconjugate of claim 41 wherein said substrate is a component of an in vitro diagnostic device.

45. The kit comprising one or more bioconjugates of claim 1 and reagents and apparatus suitable for administering said bioconjugate to an individual.

46. The kit of claim 45 wherein said bioconjugate is in a pharmaceutically acceptable carrier.

47. The biointerface formed on a mammalian tissue, wherein said biointerface comprises a plurality of bioconjugates of claim 1 bound to a plurality of ligands on said tissue.

48. A method of preparing a bioconjugate comprising the steps of:
a. providing a hydrophilic polymer having one or more reactive groups;
b. providing a bioselective peptide comprising a chemical group capable of reacting with said reactive groups; and
c. contacting said polymer and said peptide under conditions whereby said reactive and chemical groups react to form said bioconjugate.

49. The method of claim 48 wherein the reactive groups of said polymer are hydroxyl groups and the chemical group of said peptide is a sulfhydryl group.

50. The method of claim 48 wherein said polymer is a polysaccharide.

51. The method of claim 50 wherein said polysaccharide is activated dextran.

52. The method of claim 50 wherein said polysaccharide is hydroxyl starch.

53. The method of claim 50 wherein said peptide is selected from the group consisting of SEQ ID NOS 7-14, 25-32, 35-38, 43-48, 55-56, 65, 66, 93, 94, 97, 98, 107-110, 119-124, 133-136, 141, 142, 153, 154, 157-164, 171-174, 179-200, 203-212, 215 and 216, said peptide comprising a cysteine residue.

54. The method of claim 50 wherein said peptide is selected from the group consisting of SEQ ID NOS 1-218, said peptide comprising in addition an N-terminal or a C-terminal cysteine residue.

55. A method of preparing a bioconjugate comprising the steps of:
a. providing a peptide selected from the group consisting of SEQ ID NOS 1-218;
b. modifying said peptide by addition of an N-terminal or C-terminal cysteine residue;
c. providing an amount of activated dextran; and
d. contacting said activated dextran and said modified peptide under conditions, whereby said dextran and said modified peptide react to form said bioconjugate.

56. A method for preventing adhesion of a mobile cell to a cell immobilized on a substrate comprising the step of applying a bioconjugate specific for said immobilized cell under such conditions that said bioconjugate forms a cell adhesion barrier on said immobilized cell.

57. A method of blocking pathological reactions triggered by cellular interactions in a living tissue, said method comprising the step of administering to the living tissue a bioconjugate selective for a target tissue whereby the bioconjugate forms a cell adhesion barrier at a targeted tissue site.

58. The method of claim 57, wherein said bioconjugate comprises the binding portion of an integrin for a ligand expressed in said target tissue.

59. The method of claim 58 wherein said bioconjugate is administered intravascularly, orally, intramuscularly, intraperitoneally, subcutaneously, cerebrospinally, endovascularly, rectally or topically.

60. The method of claim 59 wherein said bioconjugate is administered intravascularly in a biologically compatible solution at a concentration of between about 1 μg/L and 100 g/L.

61. The method of claim 58 wherein said bioconjugate is administered to an individual in a pharmaceutically acceptable composition.

62. The method of claim 58 wherein the amount of administered bioconjugate is between about 1-1000 mg/kg body weight.

63. The method of claim 57 for preventing and treating thrombosis, wherein an anti-coagulating amount of a bioconjugate comprising one or more peptides capable of binding selectively to integrin ligands expressed on inflamed endovascular cells is administered to tissue containing said inflamed endovascular cells.

64. The method of claim 63 wherein said integrin ligands are CN I-IV, LN, or the Echovirus-1 receptor.

65. The method of claim 63 wherein said peptide is selected from the group consisting of P-2, P-49, and SEQ ID NOS 1, 2, 3-8, 91-106, 129-192, 203 and 204.

66. The method of claim 57 for preventing and treating atherosclerosis, wherein an anti-atherosclerotic effective amount of said bioconjugate comprising one or more peptides capable of binding selectively to integrin ligands expressed on or around atherosclerotic cells is administered to tissue containing said atherosclerotic cells.

67. The method of claim 66 wherein said integrin ligands are VCAM-1, FN, MAdCAM-1, TSP, invasin or a combination thereof.

68. The method of claim 66 wherein said peptide is selected from the group consisting of P-49 and SEQ ID NOS 9-38, 59-106, 129-202 and 207-210.

69. The method of claim 57 for preventing and treating systemic inflammatory response syndrome wherein an effective amount of said bioconjugate comprising one or more peptides capable of binding selectively to integrin ligands expressed on cells in inflamed tissue is administered to said tissue.

70. The method of claim 69 wherein said integrin ligands are FN, L1 or invasin.

71. The method of claim 69 wherein said bioconjugate comprises one or more peptides selected from the group consisting of P-49 and SEQ ID NOS 9-38, 59-106, 129-202 and 207-210.

72. The method of claim 58 for preventing and treating multiple organ failure wherein an failure effective amount of said bioconjugate comprising one or more peptides capable of binding selectively to integrin ligands expressed on cells in affected tissue is administered to said tissue.

73. The method of claim 72 wherein said integrin ligands are ICAM-1, ICAM-2, ICAM-3, LPS or a combination thereof.

74. The method of claim 72 wherein said bioconjugate comprises one or more peptides selected from the group consisting of P-49 and SEQ ID NOS 39-58, 107-128 and 211-218.

75. The method of claim 57 for preventing and treating autoimmune disease wherein an effective amount of a bioconjugate comprising one or more peptides capable of binding selectively to integrin ligands expressed on cells implicated in the autoimmune disease is administered to tissue containing said cells.

76. The method of claim 75 wherein said integrin ligand is VCAM-1, FN, MAdCAM-1, TSP, invasin, ICAM-1, ICAM-2, ICAM-3, LPS, iC3b, ICAM-1, ICAM-2, ICAM-4, Fb, Factor X, CD23, NIF, heparin, β-glucan, LPS, FN, Fb, CN I, VN, FN, LN, CN, Fb, Factor X, CD23, NIF, heparin, β-glucan or a combination thereof.

77. The method of claim 75 wherein said bioconjugate comprises one or more peptides selected from the group consisting of P-2, P-49 and SEQ ID NOS 1-218.

78. The method of claim 57 for preventing and treating inflammatory diseases wherein an effective amount of a bioconjugate comprising one or more peptides capable of binding selectively to integrin ligands expressed on cells of inflamed tissue is administered to a tissue containing said inflamed cells.

79. The method of claim 78 wherein said integrin ligand is CN I-IV, LN, Echovirus-1 receptor, VCAM-1, FN, MAdCAM-1, TSP, Invasin, L1, LPS, ICAM-1-4, iC3b, Fb, Factor X, CD23, NIF, heparin, β-glucan, VN, vWF or a combination thereof.

80. The method of claim 78 wherein said bioconjugate comprises one or more peptides selected from the group consisting of P-2, P-49, and SEQ ID NOS 1-202 and 205-219.

81. The method of claim 58 for preventing and treating allograft transplant rejection wherein an anti-rejection amount of a bioconjugate comprising one or more peptides capable of binding selectively to integrin ligands expressed on T cells implicated in allograft transplant rejection is administered to an individual having transplanted tissue.

82. The method of claim 81 wherein said integrin ligand is VCAM-1, FN, MAdCAM-1, TSP, invasin, ICAM-1-4, LPS, iC3b, Fb, Factor X, CD23, NIF, heparin, β-glucan, LN, CN, vWF, OP, BSP, L1 and E-cadherin.

83. The method of claim 81 wherein said bioconjugate comprises one or more peptides selected from the group consisting of P-49 and SEQ ID NOS 9-30, 39-58, 91-200 and 211-218.

84. The method of claim 81 further comprising concurrent administration of an immunosuppressant.

85. The method of claim 84 wherein said immunosuppressant is cyclosporine.

86. The method of claim 58 for preventing and treating Crohn's disease wherein an effective amount of said bioconjugate comprising one or more peptides capable of binding selectively to integrin ligands expressed on inflamed cells in gut tissue is administered to said gut tissue.

87. The method of claim 86 wherein said integrin ligand is VCAM-1, FN, MAdCAM-1, TSP, invasin, ICAM-1-4, iC3b, Fb, Factor X, CD23, NIF, heparin, β-glucan, CN I, VN, LN, OP, BSP, L1, vWF and E-cadherin.

88. The method of claim 86 wherein said bioconjugate comprises one or more peptides selected from the group consisting of P-49 and SEQ ID NOS 9-30, 30-58, 93-200 and 211-218.

89. The method of claim 58 for preventing and treating inflammatory bowel disease wherein an effective amount of a bioconjugate comprising one or more peptides capable of binding selectively to integrin ligands expressed on inflamed cells in gut tissue is administered to said gut tissue.

90. The method of claim 89 wherein said bioconjugate comprises one or more peptides selected from the group consisting of P-49 and SEQ ID NOS 9-30, 39-58, 91-200 and 21-218.

91. The method of claim 58 for preventing and treating sequelae of a bacterial infection wherein an effective amount of said bioconjugate comprising one or more peptides capable of binding selectively to integrin ligands expressed on secretory membranes is administered to said secretory membranes.

92. The method of claim 91 wherein said bioconjugate comprises one or more peptides selected from the group consisting of P-49 and SEQ ID NOS 39-58, 107-192 and 211-216.

93. The method of claim 58 for preventing and treating sepsis or septic shock, comprising administering an effective amount of a bioconjugate comprising one or more peptides capable of binding selectively to integrin ligands such as LFA-1, ICAM-1, VCAM-1 and a combination thereof.

94. The method of claim 93 wherein said bioconjugate comprises one or more peptides selected from the group consisting of P2, P-49 and SEQ ID NOS 1-30, 39-58, 91-200 and 211-18.

95. The method of claim 57 for preventing and treating ischemia-reperfusion injury, comprising administering an effective amount of a bioconjugate comprising one or more peptides capable of binding selectively to integrin ligands intravenously.

96. The method of claim 95 wherein said bioconjugate comprises one or more peptides selected from the group consisting of P-49 and SEQ ID NOS 9-30 and 39-218.

97. The method of claim 57 for preventing and treating cancer metastasis, comprising administering wherein an anti-metastasis effective amount of said bioconjugate comprising one or more peptides capable of binding selectively to integrin ligands systemically to an individual or locally to tissue containing or suspected of containing said cancer.

98. The method of claim 97, wherein said bioconjugate comprises one or more peptides selected from the group consisting of P-49 and SEQ ID NOS 91, 92, 203 and 204.

99. The method of claim 57 for treating conditions caused by viper and rattlesnake bites wherein an anti-venom effective amount of said bioconjugate comprising one or more peptides capable of binding selectively to at least one integrin ligand on a bitten tissue site is administered.

100. The method of claim 110 wherein said bioconjugate comprises a peptide having SEQ ID NOS 153 and 154.

101. Therapeutic replacement fluids comprising a bioconjugate of claim 1 and a pharmaceutically acceptable diluent.
